(19) United States
(12) Patent Application Publication
(10) Pub. No.: US 2011/0196245 A1
(43) Pub. Date: Aug. 11, 2011

(54) MEASUREMENT OF CEREBRAL HEMODYNAMIC PARAMETERS

(75) Inventors: Ben Zion Poupko, Nes Ziona (IL); Alon Rappaport, Tel-Aviv (IL); Shlomi Ben-Ari, Binyamina (IL); Boaz Shpigelman, Natania (IL)

(73) Assignee: Orsan Medical Technologies Ltd., Natania (IL)

(21) Appl. No.: 13/122,966

(22) PCT Filed: Oct. 7, 2009
(86) PCT No.: PCT/IB2009/054388

§ 371 Date: Apr. 7, 2011

Related U.S. Application Data
(60) Provisional application No. 61/103,287, filed on Oct. 7, 2008.

(51) Int. Cl. A61B 5/026; A61B 5/053

(52) U.S. Cl. 600/506; 600/547

(57) ABSTRACT

A method of finding an indication of a degree of cerebro-vascular bilateral asymmetry in a subject, comprising:
a) measuring a first impedance waveform and a second impedance waveform of the subject's head as functions of time, in each case by finding a potential difference between two voltage electrodes associated with passing a given injected current through the head between at least two current electrodes, wherein in each case the voltage electrodes are located asymmetrically on the head, or the current is injected asymmetrically into the head, or both, and wherein the locations of the voltage electrodes and the distribution of current injection in measuring the second impedance waveform are minor images of what they are in measuring the first impedance waveform; and
b) finding the indication of the degree of bilateral asymmetry from a difference between characteristics of the first and second impedance waveforms.

### RELATED APPLICATIONS

This application is related to two other PCT patent applications filed on even date, one titled “Monitoring of Acute Stroke Patients,” with attorney docket number 44064, and one titled “Diagnosis of Acute Strokes,” with attorney docket number 44066.

This application claims benefit under 35 USC 119(e) from U.S. provisional application 61/103,287, filed Oct. 7, 2008. That application is related to PCT patent application PCT/IL2007/001421, filed Nov. 15, 2007, which takes priority from U.S. patent application Ser. No. 11/610,553, filed on Dec. 14, 2006, which claims priority from, and is a continuation-in-part of, PCT patent application PCT/IB2006/050174, filed Jan. 17, 2006, which is a continuation-in-part of two related PCT patent applications PCT/IL2005/000631 and PCT/IL2005/000632, both filed Jun. 15, 2005. Those PCT applications are both continuations-in-part of U.S. patent application Ser. No. 10/893,570, filed Jul. 15, 2004, which is a continuation-in-part of PCT patent application PCT/IL03/00042, filed Jan. 15, 2003, which claims benefit under 35 USC 119(e) from U.S. provisional patent application 60/348,278, filed Jan. 15, 2002.

The contents of all of the above documents are incorporated by reference as if fully set forth herein.

### FIELD AND BACKGROUND OF THE INVENTION

The present invention, in some embodiments thereof, relates to a method and apparatus for determining cerebral hemodynamic parameters and other clinically useful data of the cerebrovascular system, and, more particularly, but not exclusively, to a method and apparatus using electrical impedance measurements.

A number of cerebral hemodynamic parameters may be clinically useful for diagnosing strokes, trauma, and other conditions that can affect the functioning of the cerebrovascular system. These parameters include regional cerebral blood volume, cerebral blood flow, cerebral perfusion pressure, mean transit time, time to peak, and intracranial pressure. Many methods that are used to measure these parameters, while giving accurate results, are not practical to use for continuous monitoring, or for initial diagnosis outside a hospital setting, because they are invasive, or because they require expensive and/or non-portable equipment. Such methods include inserting a probe into the cerebrospinal fluid or into an artery, computed tomography (CT), perfusion computed tomography (PCT), positron emission tomography (PET), magnetic resonance imaging (MRI), and transcranial Doppler ultrasound (TCD). Some of this prior art is reviewed in U.S. patent application Ser. No. 11/610,553, published as US2007/0287899 and WO2008/072223, and in the other related applications listed above.

The use of perfusion computed tomography for finding cerebral hemodynamic parameters, and the use of these parameters in evaluating and choosing courses of treatment for stroke patients, is described by Christian Baumgartner et al, “Functional Cluster Analysis of CT Perfusion Maps: A New Tool for Diagnosis of Acute Strokes,” J. of Digital Imaging 18, 219-226 (2005); by Roland Bruening, Axel Kuettner and Thomas Flohr, Protocols for Multislice CT (Springer, 2005), especially on page 96; by Ellen G. Hoeffner et al, “Cerebral Perfusion CT: Technique and Clinical Applications,” Radiology 231, 632-644 (2004); and by Hiroshi Hagiwara et al, “Predicting the Fate of Acute Ischemic Lesions Using Perfusion Computed Tomography,” J. Comput. Assist. Tomogr. 32, 645-650 (2008).

A. M. Weindling, N. Murdoch, and P. Rolfe, “Effect of electrode size on the contributions of intracranial and extracranial blood flow to the cerebral electrical impedance plethysmogram,” Med. & Biol. Eng. & Comput. 20, 545-549 (1982) describes measurements of blood flow in the head, using separate current and voltage electrodes on the front and back of the head, and measuring the peak-to-peak change in impedance over a cardiac cycle to find the blood flow. A tourniquet was placed around the head to temporarily stop the scalp blood flow, and then released, in order to determine how much of the measured blood flow was due to scalp blood flow, and how much was due to intracranial blood flow. The scalp blood flow was considered to be completely cut off when there was no detectable variation in the signal from a PPG sensor at the cardiac frequency.

J. Gronlund, J. Jalonen, and I. Valimaki, “Transcephalic electrical impedance provides a means for quantifying pulsatile cerebral blood volume changes following head-up tilt,” Early Human Development 47 (1997) 11-18, describe electrical impedance measurements of the head in premature newborn infants. Changes in impedance associated with the cardiac cycle are said to reflect changes in total cerebral blood volume, and earlier papers are referenced which are said to demonstrate this. Variability in impedance, in the range of 1.5 to 4 Hz, was found to decrease by 27%, on average, when the infants' heads were tilted up by 20 degrees. An earlier paper describing related research by the same group is J. Gronlund et al, “High Frequency Variability of Transcephalic Electrical Impedance: A New Parameter for Monitoring of Neonatal Cerebral Circulation?”, Proceedings of the Annual International Conference of the Engineering in Medicine and Biology Society, Paris, Oct. 29-Nov. 1, 1992, New York, IEEE, US, Vol. 6 Conf. 14, 29 Oct. 1992, pages 2513-2515.

Rheoencephalography (REG) is a technique that uses bio-impedance measurements of the head to obtain information on about cerebral blood circulation and circulatory problems. Generally, changes in impedance Z across the head, for a particular arrangement of electrodes, are measured as a function of time t over a cardiac cycle, and sometimes over a breathing cycle, due to changes in the volume and distribution of blood in the head. As described by W. Traczewski et al, “The Role of Computerized Rheoencephalography in the Assessment of Normal Pressure Hydrocephalus,” J. Neurotrauma 22, 836-843 (2005), REG is commonly used to measure or diagnose problems with circulatory resistance, and problems with arterial elasticity. In patients with normal pressure hydrocephalus, for example, Traczewski et al find two different patterns in Z(t), depending on the elasticity of the small cerebral arteries. The pattern of Z(t) seen in a given patient is said to be useful for making predictions about the likely outcome of different treatments for the hydrocephalus. These patients all had similar, normal values of ICP.

G. Bonmassar and S. Iwaki, “The Shape of Electrical Impedance Spectrosopy (EIS) is altered in Stroke Patients,” Proceedings of the 26^{th} Annual Conference of IEEE EMBS, San Francisco, Calif., USA, Sep. 1-5, 2004, describes a system that uses electrical impedance to measure an asymmetry in the distribution of cerebral spinal fluid that is present in stroke patients, but not in healthy volunteers. The system uses 10 electrodes placed symmetrically around the subject's head, and passes white noise current at 0 to 25 kHz between any selected pair of electrodes, while measuring the potentials at all the electrodes. The system was found to work best if current was passed between the front and back of the head, but the paper also describes passing current between symmetrically placed electrodes on the left and right sides of the head.

WO 02/071923 to Bridger et al describes measuring and analyzing pulse waveforms in the head obtained from acoustic signals. Head trauma patients, and to a lesser extent stroke patients, are found to have differences from normal subjects. Trauma and stroke patients are found to have higher amplitudes at harmonics of the heart rate, at 5 to 10 Hz, than normal subjects do.

Yu. E. Moskalenko et al, “Slow Rhythmic Oscillations within the Human Cranium: Phenomenology, Origin, and Informational Significance,” Human Physiology 27, 171-178 (2001), describes the use of electrical impedance measurements of the head, and TCD ultrasound measurements, to study slow waves, at frequencies of 0.08 to 0.2 Hz, that are apparently related to regulation of blood supply and oxygen consumption in the brain, and the circulation of cerebrospinal fluid. The studies were done with healthy subjects and with patients suffering from intracranial hypertension. A. Ragauskas et al, “Implementation of non-invasive brain physiological monitoring concepts,” Medical Engineering and Physics 25, 667-687 (2003), describe the use of ultrasound to non-invasively monitor such slow waves, as well as pulse waves at the cardiac frequency, in intracranial blood volume, in head injury patients, and find that they can be used to determine intracranial pressure.

Additional background art includes WO 02/087410 to Naisberg et al; Kidwell C S et al, Comparison of MRI and CT for detection of acute intracerebral hemorrhage. JAMA; 2004: 292: 1823-1830; Horowitz SH et al, Computed tomographic-angiographic findings within the first 5 hours of cerebral infarction, Stroke; 1991: 22 1245-1253; The ATLANTIS, ECASS, and NINDS rt-PA study group investigators, Association of outcome with early stroke treatment: Pooled analysis of ATLANTIS, ECASS, and NINDS rt-PA stroke trials, Lancet; 363: 768-774; Albers G et al, Antithrombotic and thrombolytic therapy for ischemic stroke: The seventh ACCP conference on antithrombotic and thrombolytic therapy, Chest 2004; 126: 483-512; Kohrmann M et al., MRI versus CT-based thrombolysis treatment within and beyond the 3 hour time window after stroke onset: a cohort study, Lancet Neurol 2006; 5:661-667; Albers G W et al, Magnetic resonance imaging profiles predict clinical response to early reperfusion: The diffusion and perfusion imaging evaluation for understanding stroke evolution (DEFUSE) study, Ann Neurol 2006; 60: 508-517; Johnston S C et al, National stroke association guidelines for the management of transient ischemic attacks, Ann Neurol 2006; 60: 301-313.

### SUMMARY OF THE INVENTION

An aspect of some embodiments of the invention concerns apparatus and methods for determining cerebral hemodynamic parameters, including asymmetries in the parameters, from electric impedance and/or photoplethysmographic (PPG) signals of the head and analysis of their morphologies, including measures of rise time of blood volume during a cardiac cycle, and measures of nonlinearity of blood volume as a function of time during its rise or fall.

There is thus provided, in accordance with an exemplary embodiment of the invention, a method of finding an indication of a degree of cerebrovascular bilateral asymmetry, comprising:
a) placing on the head, in a substantially bilaterally symmetric way, a set of at least three electrodes, each electrode adapted to pass current through the head to another electrode, or to measure potential relative to another electrode, or both;
b) measuring a first asymmetric impedance waveform of the head as a function of time over at least one cardiac cycle, using the electrodes;
c) measuring a second asymmetric impedance waveform of the head as a function of time over at least one cardiac cycle, using the electrodes, the second impedance measurement being a minor image of the first impedance measurement; and
d) finding the indication of the degree of bilateral asymmetry from a difference between characteristics of the first and second impedance waveforms.

Optionally, placing the set of at least three electrodes on the head comprises placing a first electrode and a second electrode symmetrically on the left and right sides of the head, and placing a third electrode substantially on the bilateral symmetry plane of the head.

Optionally, measuring the first impedance comprises measuring a voltage between the first and third electrodes when current is passed between the first and third electrodes, and measuring the second impedance comprises measuring a voltage between the second and third electrodes when current is passed between the second and third electrodes.

Alternatively or additionally, measuring the first impedance comprises measuring a voltage between the first and third electrodes when current is passed between the first and second electrodes, and measuring the second impedance comprises measuring a voltage between the second and third electrodes when current is passed between the first and second electrodes.

In an embodiment of the invention, placing the set of at least three electrodes on the head comprises placing first and second electrodes symmetrically on the left and right sides of the head, respectively, and placing third and fourth electrodes symmetrically on the left and right sides of the head, respectively, closer together than the first and second electrodes.

Optionally, the first impedance waveform is measured using the first and fourth electrodes, and the second impedance waveform is measured using the second and third electrodes, and the method also includes:
a) measuring a first surface impedance waveform using the first and third electrodes;
b) measuring a second surface impedance waveform using the second and fourth electrodes; and
c) correcting the first and second impedance measurements, to reduce a contribution of surface impedance, using the results of the first and second surface impedance measurements.

There is further provided, in accordance with an exemplary embodiment of the invention, a system for finding an indication of a degree of cerebrovascular bilateral asymmetry, comprising:
a) an electric current source;
b) a set of at least three electrodes, each electrode adapted to pass current from the current source through the head to another electrode, or to measure a potential of a location on the head relative to another electrode at a different location on the head, or both, such that, when placed symmetrically on the head, the electrodes are adapted to measure a first asymmetric impedance of the head, and a second asymmetric impedance of the head that is a minor image of the first asymmetric impedance; and
c) a controller which uses the electrodes to measure waveforms of the first and second impedances as a function of time for at least one cardiac cycle, to find characteristics of the first and second impedance waveforms, and to use a difference between characteristics of the first and second impedance waveforms to find the indication of the degree of bilateral asymmetry.

There is further provided, in accordance with an exemplary embodiment of the invention, a method of analyzing a signal obtained from electrical impedance data of the head as a function of a cardiac cycle time parameter, the method comprising:
a) determining a minimum of the signal over the cardiac cycle time;
b) determining an effective maximum of the signal over the cardiac cycle time; and
c) determining a rise interval of the cardiac cycle time, between the minimum and the effective maximum, over which the signal is rising according to a rise time criterion.

Optionally, the signal is obtained from a combination of the electrical impedance data of the head, and photoplethysmography (PPG) data of the head, as a function of the cardiac cycle time parameter.

Optionally, the signal is obtained by taking a difference or a weighted difference between an electrical impedance signal and a PPG signal, or by dividing an electrical impedance signal by a PPG signal.

There is further provided, in accordance with an exemplary embodiment of the invention, a method of analyzing an electrical impedance signal and a PPG signal of the head, comprising:
a) obtaining a measure of the impedance signal by analyzing the impedance signal according to the method of an exemplary embodiment of the invention;
b) obtaining a measure of the PPG signal by analyzing the PPG signal according the same method, but using the PPG signal in place of the signal obtained from electrical impedance data; and
c) adjusting the measure of the impedance signal using the measure of the PPG signal.

Optionally, adjusting the measure of the impedance signal comprises taking a difference or weighted difference between the measure of the impedance signal and the measure of the PPG signal, or taking a ratio of the measure of the impedance signal and the measure of the PPG signal.

Optionally, said maximum is a global maximum.

Alternatively, said maximum is a first local effective maximum, being either a first local maximum before the global maximum, or, if there is no local maximum before the global maximum, an inflection point of positive third derivative before the global maximum.

Optionally, the rise time criterion is that the rise interval begins at the time of the minimum and ends at the time of said maximum.

Alternatively, the rise time criterion is that the rise interval begins at a time where the signal is a first fixed percentage of the total range above the minimum, and the rise interval ends at a time where the signal is a second fixed percentage of the total range below said maximum.

Optioinally, the first fixed percentage is between 5% and 20%.

Optionally, the second fixed percentage is between 10% and 30%.

Optionally, the method also includes normalizing the rise interval to a cardiac cycle period.

Optionally, the method also includes monitoring changes in the rise interval in a patient.

In an embodiment of the invention, the method includes using the changes in the rise interval to monitor one or more of cerebral blood flow, cerebral blood volume, time to peak, and mean transit time.

Optionally, the method includes alerting medical personnel if the rise interval changes by 10% or more.

Optionally, determining an effective maximum of the signal comprises determining both a global maximum and a first local effective maximum of the signal.

Optionally, determining the rise interval of the cardiac cycle over which the signal is rising comprises determining a first peak rise interval over which the signal is rising between the minimum and a first local effective maximum according to a first peak rise time criterion, and determining a total rise interval over which the signal is rising between the minimum and a global maximum, according to a total rise time criterion, and also including finding a ratio of the first peak rise interval to the total rise interval.

Optionally, the method also includes finding a ratio of a height of the first local maximum above the minimum, to a height of the global maximum above the minimum.

In an embodiment of the invention, the method also comprises:
a) finding an integral of the signal over the rise interval;
b) finding an integral of the signal over the whole cardiac cycle; and
c) finding a ratio of the integral over the rise interval to the integral over the whole cardiac cycle.

There is further provided, according to an exemplary embodiment of the invention, a method of analyzing an electrical impedance signal and a PPG signal of the head, comprising:
a) obtaining a ratio of the integral over the rise interval to the integral over the whole cardiac cycle for the electrical impedance signal, according to the method of an embodiment of the invention;
b) obtaining a ratio of the integral over the rise interval to the integral over the whole cardiac cycle for the PPG signal according the same method, but using the PPG signal in place of the signal obtained from electrical impedance data; and
c) adjusting said ratio for the impedance signal using the ratio for the PPG signal.

Optionally, adjusting said ratio for the impedance signal comprises dividing by the ratio for the PPG signal.

Optionally, the method also includes using said ratio to monitor changes in one or more of cerebral blood flow, cerebral blood volume, time to peak, and mean transit time, in a patient.

Optionally, the method includes alerting medical personnel if said ratio changes by 10% or more.

There is further provided, according to an exemplary embodiment of the invention, a method of analyzing an electrical impedance signal of the head as a function of a cardiac cycle time parameter, the method comprising:
a) finding an integral of the signal over an interval comprising part or all of a cardiac cycle; and
b) finding an average value of the signal in the interval by dividing the integral by the length of the interval.

Optionally, the method also includes dividing said average value of the signal in the interval by an average of the maximum and minimum of the signal in the interval, as a measure of the convexity or concavity of the signal in the interval.

Optionally, the interval is a first peak rise interval, over which the signal is rising between the minimum and a first local effective maximum, according to a first peak rise time criterion.

Alternatively, the interval is substantially the whole cardiac cycle.

Optionally, the method also includes using the average value of the signal to estimate one or both of intracranial pressure and cerebral blood volume.

There is further provided, in accordance with an exemplary embodiment of the invention, a method of finding an indication of a degree of cerebrovascular bilateral asymmetry, comprising:
a) measuring a left surface blood flow using a first sensor in a region on the left side of the head;
b) measuring a right surface blood flow using a second sensor in a region on the right side of the head; and
c) using a difference between the left and right surface blood flows to find the indication of the degree of cerebrovascular bilateral asymmetry.

Optionally, the first and second sensors are PPG sensors.

Alternatively, the first and second sensors are surface impedance sensors.

Optionally, the method includes measuring a value of a cerebral hemodynamic parameter symmetrically across the head, and using the difference between the left and right surface blood flows comprises correcting the value of the cerebral hemodynamic parameter using the left surface blood flow, correcting the value of the cerebral hemodynamic parameter using the right surface blood flow, and using a difference between the two corrected values of the cerebral hemodynamic parameter.

Optionally, the first and second sensors are substantially identical, and the regions on the left and right sides of the head are substantially mirror images of each other around the bilateral symmetry plane of the head.

There is further provided, in accordance with an exemplary embodiment of the invention, a system for finding an indication of a degree of cerebrovascular bilateral asymmetry, comprising:
a) a first and a second sensor adapted for measuring surface blood flow on the head; and
b) a controller which uses the first and second sensors to measure surface blood flow in regions respectively on the left and right sides of the head, and to use a difference between the first and second measured blood flows to find the indication of the degree of cerebrovascular bilateral asymmetry.

Optionally, finding the indication of the degree of asymmetry comprises analyzing the first and second impedance waveforms, or a waveform derived from the first and second impedance waveforms, or both, according to the method of an embodiment of the invention.

There if further provided, according to an exemplary embodiment of the invention, a method of analyzing an electric impedance measurement of the head taken over a time interval, comprising:
a) measuring an amplitude of slow waves in the impedance signal, at frequencies between 0.08 and 0.2 Hz; and
b) normalizing the amplitude of the slow waves by an average value of the impedance during the time interval.

Optionally, finding the indication of the degree of asymmetry comprises finding a peak-to-peak height of the first and second impedance waveforms, or a waveform derived from the first and second impedance waveforms, or both.

Alternatively or additionally, finding the indication of the degree of asymmetry comprises finding a maximum slope of the first and second impedance waveforms, or a waveform derived from the first and second impedance waveform, or both.

Alternatively or additionally, finding the indication of the degree of asymmetry comprises finding an interval from a time of minimum value, to a time of maximum slope, for the first and second impedance waveforms, or a waveform derived from the first and second impedance waveforms, or both.

Alternatively or additionally, finding the indication of the degree of asymmetry comprises finding a ratio of a height of a first peak to a height of a second peak, relative to a minimum, for the first and second impedance waveforms, or a waveform derived from the first and second impedance waveforms, or both.

Optionally, the method also comprises comparing the first and second impedance waveforms to an impedance waveform of a healthy subject, and determining which side of the head an abnormality causing the asymmetry is located on, using differences between the first and second waveforms, and the waveform of the healthy subject.

Optionally, the indication of the degree of cerebrovascular bilateral asymmetry comprises a measure of severity of a pathological cerebrovascular condition.

Alternatively or additionally, the indication of the degree of asymmetry comprises a measure of a degree of asymmetry of a cerebral hemodynamic parameter.

Optionally, finding the indication of the degree of asymmetry comprises:
a) analyzing the first impedance waveform according to the method of an embodiment of the invention to find a first rise interval;
b) analyzing the second impedance waveform according to the same method to find a second rise interval; and
c) using changes in the first and second rise intervals to monitor asymmetry in one or more of cerebral blood flow, cerebral blood volume, time to peak, and mean transit time.

Optionally, finding the indication of the degree of asymmetry comprises:
a) analyzing the first impedance waveform according to the method of an embodiment of the invention to find a first ratio of integrals;
b) analyzing the second impedance waveform according to the same method to find a second ratio of integrals; and
c) using changes in the first and second ratios of integrals to monitor bilateral asymmetry in one or more of cerebral blood flow, cerebral blood volume, time to peak, and mean transit time.

There is further provided, in accordance with an exemplary embodiment of the invention, a system for obtaining and analyzing electrical impedance data of the head, comprising:
a) an electric current source;
b) a set of at least two electrodes, including at least two electrodes which pass current from the current source between them through the head, and at least two electrodes which measure a potential difference between their locations on the head, the electrodes thereby providing impedance data of the heat; and
c) a data analyzer, which finds an impedance signal as a function of phase of a cardiac cycle from the impedance data, determines a minimum of the signal over the cardiac cycle time, determines an effective maximum of the signal over the cardiac cycle time, and determines a rise interval of the cardiac cycle time, between the minimum and the effective maximum, over which the signal is rising according to a rise time criterion.

There is further provided, in accordance with an exemplary embodiment of the invention, a system for obtaining and analyzing electrical impedance data of the head, comprising:
a) an electric current source;
b) a set of at least two electrodes, including at least two electrodes which pass current from the current source between them through the head, and at least two electrodes which measure a potential difference between their locations on the head, the electrodes thereby providing impedance data of the heat; and
c) a data analyzer, which finds an impedance signal as a function of phase of a cardiac cycle from the impedance data, finds an integral of the signal over an interval comprising part or all of a cardiac cycle, and finds an average value of the signal in the interval by dividing the integral by the length of the interval.

There is further provided, according to an exemplary embodiment of the invention, a method of finding an indication of a degree of cerebro-vascular bilateral asymmetry in a subject, comprising:
a) measuring a first impedance waveform and a second impedance waveform of the subject's head as functions of time, in each case by finding a potential difference between two voltage electrodes associated with passing a given injected current through the head between at least two current electrodes, wherein in each case the voltage electrodes are located asymmetrically on the head, or the current is injected asymmetrically into the head, or both, and wherein the locations of the voltage electrodes and the distribution of current injection in measuring the second impedance waveform are minor images of what they are in measuring the first impedance waveform; and
b) finding the indication of the degree of bilateral asymmetry from a difference between characteristics of the first and second impedance waveforms.

Optionally, the electrodes used for measuring the first and second impedance waveforms comprise at least three electrodes, and the method also includes placing the at least three electrodes on the head in a bilaterally symmetric configuration before measuring the first and second impedance waveforms.

Optionally, measuring the first impedance waveform comprises finding the potential difference between a first voltage electrode placed on a temple of the subject, and a second voltage electrode placed on the head behind the ear, on a same side of the head as the first voltage electrode.

Optionally, measuring the first impedance waveform comprises finding the potential difference between the first and second voltage electrodes while passing the current through the head between a first and a second current electrode, the first current electrode being comprised in a same structure as, or placed adjacent to, the first voltage electrode, and the second current electrode being comprised in a same structure as, or placed adjacent to, the second voltage electrode.

There is further provided, according to an exemplary embodiment of the invention, a system for finding an indication of a degree of cerebrovascular bilateral asymmetry, comprising:
a) an electric current source;
b) a voltmeter that measures potential differences between two electrodes;
c) a set of at least three electrodes, at least three of them adapted to pass current from the current source through the head, and at least three of them adapted to be used by the voltmeter for measuring a potential difference between different locations on the head; and
d) a controller which, when the electrodes are placed appropriately on the head, makes a first impedance measurement by using a first asymmetrically placed subset of the electrodes to measure the voltage associated with a given current passed through the head, uses a second subset of the electrodes to make a second impedance measurement that is a minor image of the first impedance measurement, and uses a difference between characteristics of waveforms of the first and second impedance measurements to find the indication of the degree of bilateral symmetry.

There is further provided, according to an exemplary embodiment of the invention, a method of finding an indication of a degree of cerebrovascular bilateral asymmetry in a subject, comprising:
a) measuring a characteristic of surface blood flow on the left side of the subject's head, using at least a first sensor in a region on the left side of the head;
b) measuring a characteristic of surface blood flow on the right side of the subject's head, using at least a second sensor in a region on the right side of the head; and
c) using a difference between the characteristics of the surface blood flows on the left and right sides of the head to find the indication of the degree of cerebrovascular bilateral asymmetry.

Optionally, the characteristic of surface blood flow on each side of the head comprises a phase difference in a pulse waveform between the surface blood flow on that side of the head and blood flow in a major artery on the same side of the subject's neck, and measuring said characteristic comprises measuring the pulse waveform of the surface blood flow using the sensor in the region on that side of the head, and measuring the pulse waveform of the blood flow in the major artery on that side of the neck, using an artery blood flow sensor adjacent to the major artery on that side of the neck.

Optionally, the artery blood flow sensors on the left and right sides of the neck each comprise PPG sensors.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions, processed signals, and/or raw data, and optionally a non-volatile storage, for example, a magnetic hard-disk and/or removable media, for storing instructions, processed signals, and/or raw data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic view of a subject with electrodes for impedance measurements and PPG sensors placed on his head, according to an exemplary embodiment of the invention;

FIGS. 2A and 2B are schematic views of a subject with electrodes placed on his head for impedance measurements, according to two other exemplary embodiments of the invention;

FIG. 2C is a schematic view of a subject with PPG sensors placed on his head and neck for measurements of blood circulation, according to another exemplary embodiment of the invention;

FIG. 3 is a schematic plot of impedance Z as a function of time in a cardiac cycle, measured by electrodes placed on the head, showing rise intervals defined in different ways, according to an exemplary embodiment of the invention;

FIG. 4 is a schematic plot of impedance Z as a function of time in a cardiac cycle, showing an effective first local maximum defined by an inflection point, according to an exemplary embodiment of the invention; and

FIGS. 5A, 5B and 5C are schematic plots of impedance Z as a function of time in a cardiac cycle, showing rising and falling intervals that are linear, concave, and convex, according to an exemplary embodiment of the invention; and

FIG. 6 is a schematic plot showing the correlation between normalized slow wave amplitude and the volume of stroke lesions in ischemic stroke patients, according to an exemplary embodiment of the invention.

### DESCRIPTION OF EMBODIMENTS OF THE INVENTION

The present invention, in some embodiments thereof, relates to a method and apparatus for determining cerebral hemodynamic parameters and, more particularly, but not exclusively, to a method and apparatus using electrical impedance measurements.

An aspect of some embodiments of the invention concerns a method and apparatus for determining an indication of a degree of cerebrovascular bilateral asymmetry. The indication of the degree of asymmetry may be a measure of asymmetry in a cerebral hemodynamic parameter, such as regional cerebral blood flow, which can be important for diagnosing and monitoring strokes and other medical conditions. Additionally or alternatively, the indication of the degree of asymmetry may itself be a measure of a medical condition, such as the volume of a stroke lesion on one side of the head.

In an exemplary embodiment of the invention, electrodes are applied to the head, and are used to make two minor image measurements of electrical impedance signals on the left and right sides of the head, as a function of time. As used herein, “two mirror image measurements of electrical impedance” means two measurements of a difference in potential associated with a given distribution of current injected into the head, in which both the locations of the electrodes for measuring the potential difference, and the distribution of injected current, are minor images with respect to the bilateral symmetry plane of the head. The electrodes used for measuring the potential difference may also be used for injecting the current, or different electrodes may be used. For the two mirror image measurements to be different, either the placement of the electrodes for measuring the potential difference, or the distribution of injected current, or both, is asymmetric in each measurement, with respect to the bilateral symmetry of the head. Optionally, all the electrodes needed for both measurements are put in place before the measurements are made and not moved between measurements, with the complete set of electrodes being arranged symmetrically with respect to the bilateral symmetry of the head, and with at least three electrodes placed on the head in order to make two independent measurements that are minor images of each other. Optionally, for each measurement, the potential difference is measured between electrodes placed on the temple and behind the ear on the same side of the head, and optionally the current is also injected between electrodes placed on the temple and behind the ear on that side of the head.

The impedance measurements are optionally made over an interval of one or more cardiac periods. The impedance waveform as a function of time typically has a characteristic dependence on the phase of the cardiac cycle, which approximately repeats for each cardiac cycle. Measuring the impedance over at least one cardiac cycle makes it possible to find the entire cardiac cycle waveform, and measuring the impedance over more than one cardiac cycle makes it possible to reduce noise by finding the waveform as a function of cardiac cycle phase, averaged over more than one cardiac cycle. In addition, the impedance may exhibit slow waves at frequencies below the cardiac cycle frequency, which can be observed if the impedance is measured over at least a characteristic slow wave period. An impedance measurement made over one or more time intervals shorter than a cardiac period can also be useful, particularly if the measurement is gated to the cardiac cycle, for example if it is only desired to analyze the impedance during a particular part of the cardiac cycle, such as the rise time, or if it is desired to measure only the slow waves and to eliminate the cardiac cycle waveforms from the signal by gating to the cardiac cycle.

Differences between characteristics of the waveforms of the two impedance signals, such as rise time of the cardiac cycle, or slow wave amplitude, are used to determine the indication of the degree of cerebrovascular bilateral asymmetry. Optionally, if an asymmetry is found, the two waveforms are each compared to a waveform from a healthy subject, in order to determine on which side of the head an abnormal condition is located. Optionally, photoplethysmography (PPG) sensors, located for example symmetrically on the left and right sides of the head, produce PPG signals which are combined with the two impedance signals to produce adjusted impedance signals, to determine the degree of asymmetry of the cerebral hemodynamic parameter. Optionally, the PPG signals are used to reduce the contribution of surface blood flow to the adjusted impedance signals.

As used herein, describing a distribution of injected current in the head as asymmetric means that the distribution of injected current is neither symmetric or anti-symmetric with respect to the bilateral symmetry plane of the head. Current flowing from an electrode placed on the center of the forehead to an electrode placed on the center of the back of the head, for example, would have a symmetric distribution of injected current, while current flowing from an electrode placed on the left temple to an electrode placed symmetrically on the right temple would have an anti-symmetric distribution of injected current. Either of those distributions of injected current would be its own mirror image, aside from a 180 degree phase shift, and would not be referred to herein as an asymmetric distribution of injected current.

It should be understood that, when it is stated that two impedance measurements are mirror images of each other, or that two electrodes used for minor image measurements are placed symmetrically on the head or neck, the distribution of injected current and the placement of the electrodes need not be precisely mirror images for the two measurements. However, the current electrodes used for injecting current, and the voltage electrodes used for measuring potential differences, are placed close enough to symmetrically with respect to the bilateral symmetry plane of the head, so that any differences in the two mirror image impedance measurements, for a healthy subject, are small, for example by a factor of at least 2, or 5, or 10, or 20, compared to the differences that would be seen in a subject with a clinically significant asymmetry in blood circulation in the head. The requisite precision in the placement of electrodes may be found by testing to see what changes in the various measures based on IPG and PPG signals, described in the Examples below, occur as a result of misplacement of the electrodes, and comparing these changes to the range that these measures exhibit over a random sample of ischemic stroke patients, typically a factor of about 2. For example, corresponding electrodes are placed within 2 cm, or 1 cm, or 5 mm, or 2 mm, or 1 mm of being at mirror image locations of each other, and electrodes that are said to be located at the bilateral symmetry plane of the head have their centers within these distances of the bilateral symmetry plane. It should be understood, of course, that all people have slight asymmetries in the external anatomy of their heads, so there is a limit to the precision to which it makes sense to talk about placing electrodes symmetrically. In rare cases, people may have grossly asymmetric brains or scalps, due to past injuries or surgery, and it may not make sense to talk about symmetric placement of electrodes, and mirror image measurements, at all, for those people. These remarks apply also to the placement of PPG sensors that are said to be placed symmetrically on the head or neck, to provide minor image PPG measurements.

As noted above, whenever two minor image measurements are described, the electrodes or sensors for both measurements may be placed on the subject before either measurement is made, with the entire set of electrodes or sensors for both measurements being arranged symmetrically. Alternatively, the electrodes or sensors for a first measurement may be placed on the subject, and after the first measurement is made, some or all of those electrodes or sensors may be removed, and the electrodes or sensors for the second measurement may then be placed on the subject, possibly using some or all of the same electrodes or sensors over again, before the second measurement is made. However, it is potentially advantageous to place all of the electrodes and sensors on the subject before either set of measurements is made, for example in order to make both measurements repeatedly over a period of time, and in the drawings showing different systems for measuring a degree of cerebrovascular bilateral asymmetry, all of the electrodes and sensors are shown in place at the same time.

It should be understood that, in a measurement of impedance, the current may be held fixed and the potential difference measured, or the potential difference may be held fixed and the current measured. In general, current or potential difference or any combination of them may be held fixed, while current or potential difference or any combination of them (but not the same quantity that is held fixed) may be measured. As used herein, “measuring potential difference for a given current,” and similar expressions, include all of these procedures, because all of them reveal what the potential difference would be for a given current. In practice, for safety reasons, it is usual to measure the potential difference while holding the current fixed at a safe level, for example no greater than 2.5 mA, or 1 mA, or 0.5 mA, or 0.2 mA, or 0.1 mA, at a frequency of at least 3 kHz, 5 kHz, 10 kHz, 20 kHz, 30 kHz, or 50 kHz.

An aspect of some embodiments of the invention concerns determining an indication of a degree of cerebrovascular bilateral asymmetry, by measuring asymmetry in surface blood flow of the head, for example using photoplethysmography (PPG), or surface electrical impedance plethysmography (surface IPG). Because a decrease in blood flow on one side of the brain, due for example to a cerebral thrombosis, is often accompanied by an increase in surface blood flow on that side of the head, comparing surface blood flow on the left and right sides of the head can reveal asymmetry in cerebral blood flow and other cerebral hemodynamic parameters. Optionally, impedance measurements of the head that are sensitive to cerebral blood flow and/or cerebral blood volume are also used to help determine the asymmetry. Optionally, blood flow is also measured in the carotid arteries on the left and right sides of the head, for example with a PPG sensor placed on the neck over each carotid artery, and compared to measurements of surface blood flow on the left and right sides of the head, for example in order to measure a phase delay in the pulse cycle between the carotid artery and the surface arteries of the head, on each side of the head.

An aspect of some embodiments of the invention concerns a method of analyzing an impedance signal of the head, in which a rise interval is measured. A rise interval is an interval of time during which the impedance signal (conventionally the negative of the impedance) is rising during a cardiac cycle. The rise interval may represent a total rise time from the minimum in the signal, at the diastole, to the global maximum in the signal, at the systole. Alternatively, the rise interval may represent a first peak rise time, from the minimum in the signal to a first local maximum before the global maximum, or, if there is no local maximum before the global maximum, then to a first local effective maximum at an inflection point where the third derivative is positive. Optionally, the rise interval is measured from the actual minimum in the signal to a maximum in the signal, whether the global maximum or first local effective maximum. Alternatively, to provide a more robust value for the rise interval, the interval is only measured in a middle part where the signal is sufficiently above the minimum, for example by 10% of the total range of the signal, and sufficiently below the maximum, for example by 20% of the total range. Optionally, the rise interval is normalized by the period of the cardiac cycle. Optionally, the ratio of the total rise time to the first peak rise time is found. Optionally, an integral of the signal over the rise interval is found. Optionally, the integral over the rise interval is normalized to an integral of the signal over the cardiac cycle.

An aspect of some embodiments of the invention concerns a method of analyzing an impedance signal of the head, as a function of time during a cardiac cycle, in which an average value of the signal is found over an interval. Optionally, the interval is the entire cardiac cycle. Alternatively, the interval is a rise interval, for example a total rise time or a first peak rise time. Optionally, the average of the signal is compared to an average of the maximum and minimum of the signal in the interval, giving an indication of the nonlinearity of the signal (whether it is linear, concave, or convex) during its rise and/or fall.

An aspect of some embodiments of the invention concerns a method of analyzing an impedance signal of the head, in which an amplitude of slow waves in the impedance, for example at frequencies of 0.08 to 0.2 Hz, is normalized to an average value of the impedance during the measurement.

Optionally, any of the methods described above for analyzing a signal obtained from impedance data, are used additionally or alternatively for analyzing a PPG signal, or for analyzing a signal that is a combination of an impedance signal and a PPG signal, for example a difference between appropriately normalized impedance and PPG signals, or a ratio of one to the other. As used herein, a “signal obtained from impedance data” can include such a combined signal, as well as a pure impedance signal that does not incorporate PPG data.

Optionally, for any of the methods of analyzing the signals, the results of the analysis are used to estimate a cerebral hemodynamic parameter of clinical interest, which is correlated with it. For example, the robust rise interval as defined above, normalized to the cardiac cycle period, has been found by the inventors to be correlated with cerebral blood flow and mean transit time (MTT), as measured by perfusion CT scans. The integral of the impedance signal during the rise interval, normalized to the integral over the cardiac cycle, has been found to be correlated with the size of a stroke lesion, as measured by a CT scan. Even better correlation has been found when this normalized integral of the impedance signal is divided by the same normalized integral of the PPG signal. As another example, the slow wave amplitude, normalized to an average value of the impedance, has been found by the inventors to be negatively correlated with the volume of stroke lesions in stroke patients.

Optionally, these or any other measures resulting from the analysis are monitored in acute stroke patients, in a hospital or home setting, with medical personnel alerted if the measure changes by 10%, or by 20%, or by 30%, particularly if it changes in a direction indicating a worsening of the patient's condition. Additionally or alternatively, medical personnel are alerted if the measure crosses a pre-defined threshold value. The alerting is done, for example, by sounding an alarm or causing a light to flash at the patient's bed and/or at a nurses' station in a hospital, or summoning an ambulance if the patient is at home.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Configuration of Electrodes and PPG Sensors

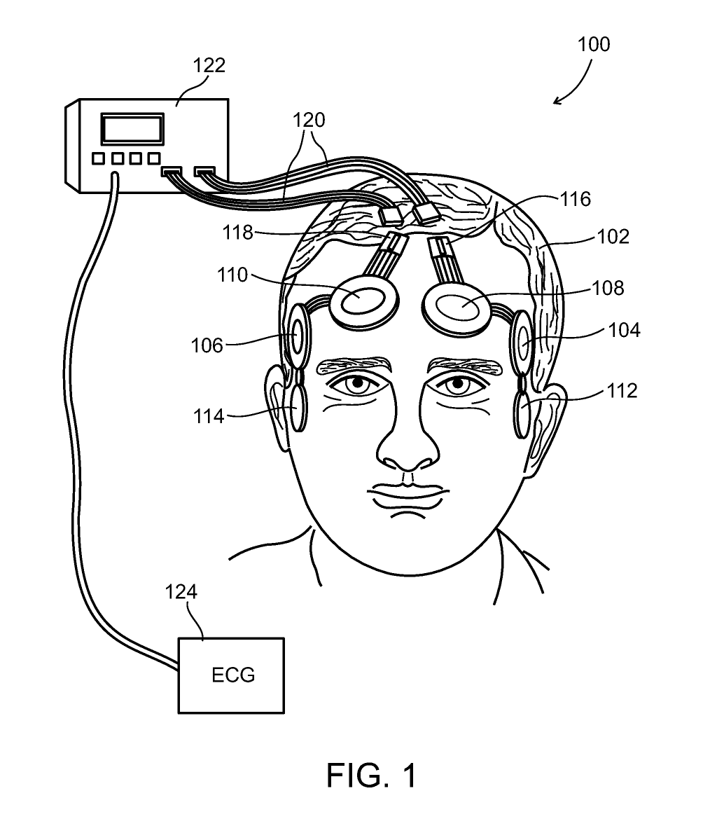

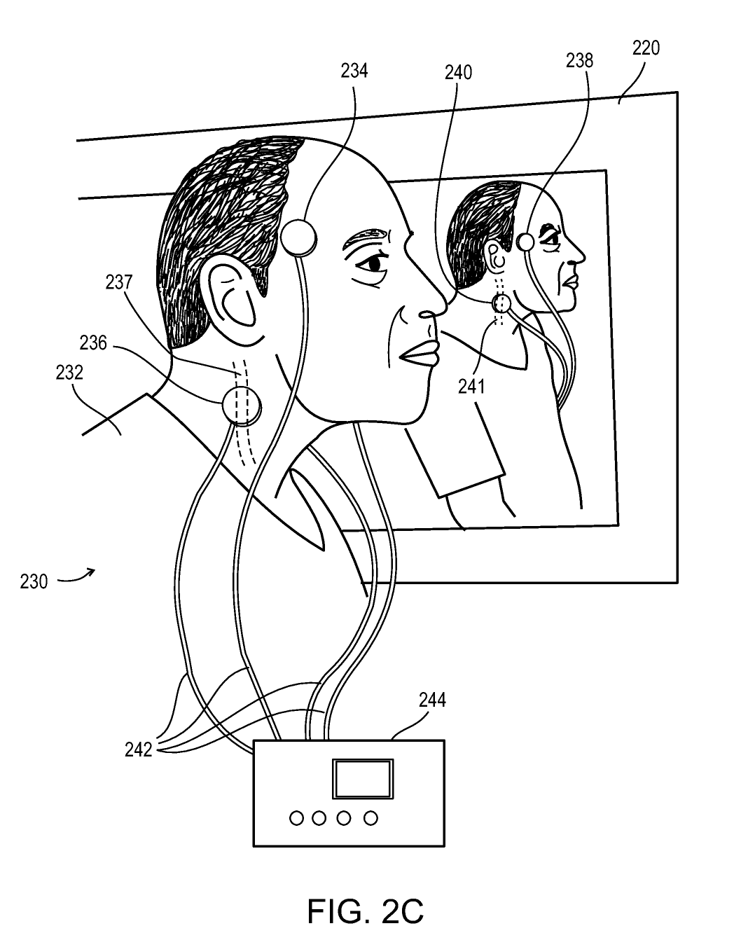

Referring now to the drawings, FIG. 1 illustrates a system 100 for finding cerebral hemodynamic parameters, mounted on a head 102 of a subject. The system comprises electrodes which are mounted symmetrically on the subject's head with respect to the bilateral symmetry plane. Electrodes 104 and 106 are mounted respectively on the subject's left and right temples, while electrodes 108 and 110 are mounted on the left and right sides of the subject's forehead, closer together than electrodes 104 and 106. Optionally, there are also PPG sensors 112 and 114, mounted on the left and right sides of the subject's head. Connectors 116 and 118 connect to cables 120, or a single cable, which connect system 100 to a controller 122. Controller 122 controls the operation of the system, and includes a power supply which provides electrical power for the electrodes and PPG sensors, and records and analyzes data from the electrodes and PPG sensors. In some embodiments of the invention, one or more of the control functions, the power supply and data analyzer may be located in separate units, rather than all being located in controller 122. Optionally, system 100 also includes an electrocardiogram (ECG) 124, which takes an ECG signal from the subject while impedance data is being taken. The ECG signal is optionally used by the data analyzer in aligning impedance or PPG signals from different cardiac cycles, and in other ways, as will be explained below in the description of FIG. 3.

It should be noted that, for any of the electrodes shown in FIG. 1 or found in other embodiments of the invention, each electrode structure optionally comprises separate current and voltage electrodes, respectively for injecting current into the head and for measuring electric potential on the surface of the head. Any configuration for separate current and voltage electrodes may be used, including, for example, the annular electrodes and interleaved spiral electrodes described in U.S. patent application Ser. No. 10/893,570, and in PCT patent application PCT/IL2005/000631, cited above as related applications, as well as other configurations known in the art. It should be understood that such a composite electrode structure is sometimes referred to herein simply as an electrode. Using separate voltage and current electrodes has the potential advantage that the measured impedance may be less dependent on the high impedance of the epidermis, and more sensitive to the impedance of the inside of the head. Alternatively, one or more of electrode structures consists of only a single electrode which is used both for measuring potential and for injecting current. An electrode structure may also consist of only a voltage electrode, or only a current electrode, with such voltage and current electrodes placed at separate locations on the head.

In an exemplary mode of operation of system 100, electric current is passed between electrodes 104 and 106, and the impedance is measured. For many of the impedance measurements described in the present application, it is primarily the change in impedance with the phase of the cardiac cycle, over one or more cardiac cycles, that is of interest. But in some cases, notably the measurement of slow wave amplitude, changes in the impedance with time, over periods longer than a cardiac cycle, are of interest. A substantial fraction of the current passed between electrodes 104 and 106 will travel through the interior of the skull, particularly because these electrodes are located on the temples, where the skull is relatively thin. However, a significant fraction of the current will also travel along the scalp, because of the relatively high impedance of the skull. In order to determine cerebral hemodynamic parameters using impedance measurements, it is generally desirable for the measurement to be able to distinguish the impedance of the interior of the skull, which depends largely on the volume of blood and cerebrospinal fluid in the brain, from the impedance of the scalp, which depends largely on the volume of blood in the scalp. In this mode of operation, in order to reduce the sensitivity of the measured impedance to the impedance of the scalp, a second impedance measurement is optionally made, termed “surface impedance,” passing current between electrodes 108 and 110. This current, unlike the current between electrodes 104 and 106, goes almost entirely through the scalp, because electrodes 108 and 110 are relatively close together, and because they are located on the forehead where the skull is relatively thick. This surface impedance measured between electrodes 108 and 110 can be used to correct the impedance measured between electrodes 104 and 106 for the scalp contribution, resulting in a value for cerebral impedance that is relatively insensitive to the impedance of the scalp. This can be done, for example, by subtracting the surface impedance from the impedance measured between electrodes 104 and 106, or by taking the ratio of the impedance measured between electrodes 104 and 106 to the surface impedance, similar to the methods for using PPG signals to correct for scalp blood flow, described in the related PCT applications PCT/IL2005/000632 and PCT/IB2006/050174. Alternatively, for example, as will be described below, an impedance signal measured between electrodes 104 and 106 is analyzed, finding one or more measures characterizing the morphology of the waveform as a function of time, and similar measures are found from the waveform of the surface impedance or PPG signal, which are then used to correct the measures found from the impedance signal measured between electrodes 104 and 106, so that they are less sensitive to surface blood flow and more dependent on cerebral hemodynamic parameters.

Optionally, the two impedance measurements are done consecutively. Alternatively they are done simultaneously, optionally using different AC frequencies so that the two measurements do not interfere with each other. It should be noted that, for safety reasons, medical measurements of impedance are generally done with AC current, at frequencies between about 10 kHz and several tens of kHz. Making the measurements simultaneously has the potential advantage that the two impedance measurements can be compared for the same cardiac cycle or cycles. If the two impedance measurements are made at different frequencies, then it is potentially advantageous that the two frequencies be close enough together so that difference in impedance is primarily due to the difference in location of the electrodes, and not to the difference in frequency. The impedance may depend on frequency because, for example, at higher frequency more of the current can go through the interior of cells, due to the lower impedance of cell membranes, which act like capacitors, while at lower frequency more of the current goes through extracellular fluid, such as the interiors of blood vessels. The current path through the interior of cells starts to become dominant above about 100 kHz.

Another mode of operation of system 100, also using four electrodes optionally at the same positions as shown in FIG. 1, is used to detect an indication of a degree of cerebrovascular bilateral asymmetry, for example an asymmetry in one or more cerebral hemodynamic parameters that may be indicative of a stroke or other cerebrovascular pathology. In this mode of operation, a first impedance is measured using at least two electrodes that are not placed symmetrically on the head with respect to each other, and a second impedance is measured using electrodes that are a minor image of the electrodes used for the first impedance. Signals of the first and second impedances, as functions of time over a cardiac cycle, may be analyzed, and one or more measures of signal morphology may be found, for each impedance signal. In a healthy subject, the pattern of blood flow and blood volume in the brain will generally be bilaterally symmetric, so the two impedance signals, and their corresponding measures of signal morphology, will be nearly the same. Differences between the two impedance measurements, or between their measures of signal morphology, may be used for diagnosing cerebrovascular abnormalities.

For example, the first impedance is measured between electrodes 104 and 110, giving an impedance measurement that depends more on the impedance of the left side of the head, and the second impedance is measured between electrodes 106 and 108, giving a measurement that is a mirror image of the first measurement, and depends more on the impedance of the right side of the head. Alternatively, the first impedance is measured between electrodes 104 and 108, and the second impedance is measured between electrodes 106 and 110. Measuring the impedance between electrodes 104 and 110, and between electrodes 106 and 108, has the potential advantage that more of the current will flow inside the skull, and less of the current will flow through the scalp, than if the impedance is measured between electrodes 104 and 108, and between electrodes 106 and 110. In some embodiments of the invention, the impedance measurement between electrodes 104 and 108 is used to correct the impedance measurement between electrodes 104 and 110 for the scalp contribution, and the impedance measurement between electrodes 106 and 110 is used to correct the impedance measurement between electrodes 106 and 108 for the scalp contribution. Alternatively, corrections for the scalp contribution on the left side of the head are made using data from PPG sensor 112, and corrections for scalp contribution on the right side of the head are made using data from PPG sensor 114.

In other modes of operation, different pairs of electrodes are used for passing current, than are used for measuring voltage differences, in some or all impedance measurements. As used herein, “voltage” or “voltage difference” is synonymous with “potential difference,” and “passing current” is synonymous with “injecting current.” Any combination of electrodes may be used for passing current and measuring voltage, as long as the configuration is asymmetric, and a minor image of the configuration for the first impedance measurement is used for the second impedance measurement. For example, current is passed between electrodes 104 and 106 for both impedance measurements, but for the first impedance measurement, voltage is measured between electrodes 104 and 110, and for the second impedance measurement, voltage is measured between electrodes 106 and 108. Other possible combinations of electrodes for passing current and measuring voltage will be apparent.

In another scenario, the first impedance measurement passes current and/or measures voltage between electrode 104 and one or both of electrodes 106 and 110, while the second impedance measurement, a mirror image of the first impedance measurement, passes current and/or measures voltage between electrode 106 and one or both of electrodes 104 and 108. In addition, in this scenario, a first surface impedance measurement is taken, utilizing at least electrodes 104 and 108, and a second surface impedance measurement is taken, utilizing at least electrodes 106 and 110. The first surface impedance measurement serves to reduce the contribution of surface impedance to the first impedance measurement, and the second surface impedance measurement serves to reduce the contribution of surface impedance to the second impedance measurement.

As used herein, a pair of relatively nearby electrodes used to measure a surface impedance, such as electrodes 104 and 108, or electrodes 106 and 110, in the examples described above, is referred to as a “surface impedance sensor,” notwithstanding the fact that any of these electrodes individually may also function to measure cerebral impedance, when used in combination with different electrodes that are further away, and notwithstanding the fact that cerebral impedance generally makes some contribution to the impedance measured even by a pair of relatively nearby electrodes on the head.

In another mode of operation, indications of a degree of cerebrovascular asymmetry between the left and right sides of the head are detected by directly comparing the signals from PPG sensors 112 and 114, which measure surface blood flow on the left and right sides of the head. Surface blood flow on one side of the head may be negatively correlated with intracranial blood flow on the same side of the head, since the carotid artery on each side of the head splits into an internal carotid artery and an external carotid artery. A blockage of the right internal carotid artery, for example, can cause more of the blood from the right carotid artery to flow through the right external carotid artery, through the scalp and skin, producing more surface blood flow and a stronger PPG signal on the right side of the head than on the left side of the head, where there is no blockage. Thus, a difference between signals from PPG sensors symmetrically placed on the left and right sides of the head can itself be indicative of a stroke or another cerebrovascular problem. Optionally, a difference between PPG signals is used together with a difference in impedance measurements, on the left and right sides of the heads, to diagnose cerebrovascular problems, or together with one or more symmetric measurements of impedance, for example between electrodes 104 and 106. For example, a PPG signal from the left side of the head is subtracted from, or divided into, a symmetric IPG signal to obtain information about cerebral hemodynamic parameters on the left side, and a PPG signal from the right side of the head is subtracted from, or divided into, the same symmetric IPG signal to obtain information about cerebral hemodynamic parameters on the right side. Alternatively, a difference in left side and right side PPG signals alone is used for this purpose, even in a system which does not use impedance measurements at all.

Any of the modes of operation of system 100 described above may be used separately or in combination with any other mode of operation, either consecutively or simultaneously. When two different modes of operation are used simultaneously, different frequencies are optionally used so that the two measurements do not interfere with each other.

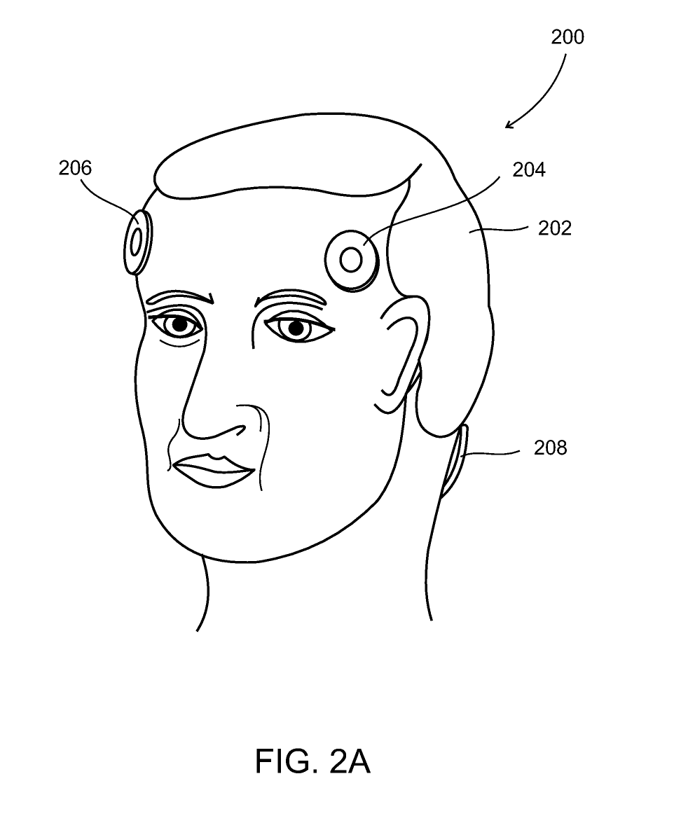

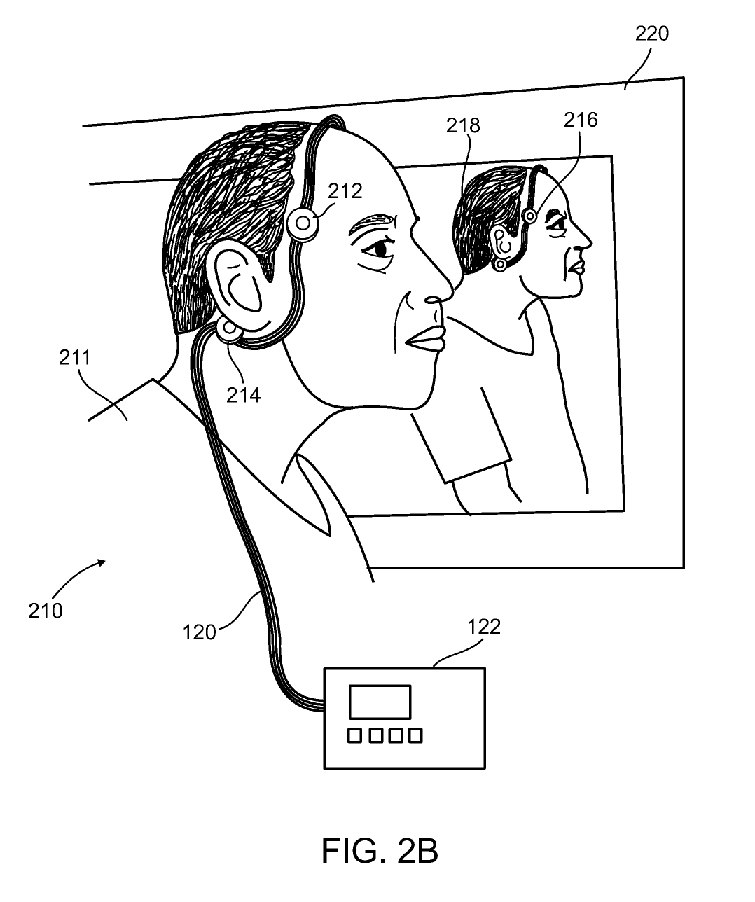

FIG. 2A shows a system 200 for measuring an indication of a degree of cerebrovascular bilateral asymmetry in a subject 202, according to another embodiment of the invention. System 200 comprises an electrode 204 mounted on the left side of the subject's head, for example at the temple, an electrode 206 mounted at a position on the right side of the head that is substantially a minor image of the position of electrode 204 on the left side, and an electrode 208 mounted substantially at the bilateral symmetry plane of the subject's head, for example at the back of the head near the foramen magnum. Although other locations on the head could be used for these electrodes, these locations have the potential advantage that they are all at thin parts of the skull, or near openings in the skull, resulting in relatively more current going through the inside of skull, and less current going through the scalp, than if the electrodes were located near thicker parts of the skull and not near openings in the skull.

System 200, like system 100, optionally has a controller, power supply and data analyzer, and cables connecting them to the electrodes, but for clarity these are not shown in FIG. 2A. Optionally, system 200 also includes PPG sensors, an ECG, and other features of system 100.

In an exemplary mode of operation of system 200, current is passed between electrodes 204 and 206, while voltage is measured between electrodes 204 and 208, and between electrodes 206 and 208. If the impedance of the subject's head is bilaterally symmetric, as it normally would be in a healthy subject, then the waveform of the voltage measured between electrodes 204 and 208 would be almost the same as the waveform of the voltage measured between electrodes 206 and 208. Any difference between these two waveforms would be an indication of an asymmetry in the impedance of the head, and a possible indication of an abnormal asymmetry in a cerebral hemodynamic parameter, such as blood flow, and a possible indication of the severity of a clinical condition, such as the volume of a stroke lesion on one side of the head.

FIG. 2B shows a system 210 for measuring an indication of a degree of cerebrovascular bilateral asymmetry in a subject 211, according to another embodiment of the invention, with a different configuration of electrodes than system 100 and system 200. Some tests by the inventors suggest that this is an advantageous configuration to use, with good sensitivity to cerebrovascular bilateral asymmetry. An electrode 212 is placed on the subject's right temple, and an electrode 214 is placed behind the subject's right ear, for example at the location shown in the drawing. An electrode 216 is placed on the subject's left temple, and an electrode 218 is placed behind the subject's left ear, which are visible in mirror 220. The electrodes are placed symmetrically on the two sides of the head. A cable 120 connects each of the electrodes to a controller 122. Although FIG. 2B shows a single multi-wire cable 120 with the electrodes daisy-chained along it, two or more separate cables may instead be used to connect the electrodes to the controller 122. The different functions ascribed to controller 122 are optionally divided among separate pieces of hardware, and this is true as well for controllers in other embodiments of the invention.

To measure an indication of a degree of cerebrovasular bilateral asymmetry, controller 122 makes a first impedance measurement, by measuring the voltage between electrodes 212 and 214, associated with a given current passing between electrodes 212 and 214. Controller 122 also makes a second impedance measurement, a mirror image of the first impedance measurement, by measuring the voltage between electrodes 216 and 218, associated with a given current passing between electrodes 216 and 218. The two measurements are made consecutively or simultaneously, and if they are made simultaneously, different frequencies are optionally used for the two measurements. Alternatively, the same frequency is used on the two sides, and the current applied on one side of the head is expected to have relatively little effect on the voltage measured on the other side of the head.

By comparing the impedance measurements made on the two sides of the head, and in particular by comparing characteristics of impedance waveforms measured as a function of time, optionally over one or more cardiac cycles, as described below, controller 122 estimates a degree of asymmetry in cerebral blood circulation, which may be clinically useful for diagnosing strokes, for example. It should be noted that, because electrodes 212 and 214 are fairly close to each other, one might expect a substantial part of the current between electrodes 212 and 214 to flow through the scalp rather than inside the cranium. Nevertheless, the inventors have found that the electrode configuration shown in FIG. 2B is useful for measuring a degree of asymmetry in cerebral blood circulation.

Electrodes 212, 214, 216 and 218, as shown in FIG. 2B, are each used both for measuring potential and for injecting current into the head. As explained elsewhere, it is potentially advantageous if each electrode comprises separate voltage and current elements, insulated from each other, for measuring potential and for injecting current. Alternatively, one or more of electrodes 212, 214, 216 and 218 may be replaced by separate current and voltage electrodes, not combined in a single structure, but optionally placed adjacent to each other on the subject's head, at the locations shown. For example the current and voltage electrodes are placed within 5 cm of each, or within 2 cm of each other, or within 1 cm of each other, at each location for which separate voltage and current electrodes are used.

The electrode configuration shown in FIG. 2B may also be used for measuring voltage on each side of the head, with a same symmetric distribution of current injected into the head during both measurements. For example, current may be passed between two electrodes, not shown in FIG. 2B, located at the bilateral symmetry plane of the head, for example an electrode placed at the center of the forehead, and an electrode placed at the back of the head, similar to electrode 208 in FIG. 2A, while electrodes 212, 214, 216 and 218 are used only for measuring voltage.

In some embodiments of the invention, a first local impedance measurement is made on the right side of the head by measuring voltage for a given current between electrodes 212 and 214, a second local impedance measurement is made on the left side of the head by measuring voltage for a given current between electrodes 216 and 218, while a third, global, impedance measurement is made by measuring voltage while passing current between an additional pair of electrodes, one on the right side and one on the left side of the head, for example electrodes at the locations of electrodes 104 and 106 in FIG. 1, or at the locations of electrodes 204 and 206 in FIG. 2A. Optionally, the three impedance measurements are made simultaneously, using three different frequencies, for example, to avoid having the different measurements interfering with each other. Alternatively, the three measurements are made at different times, for example during different cardiac cycles, or during different time slots each of much shorter duration than the shortest time scale over which the impedance changes during a cardiac cycle. Optionally, there are also one or more PPG sensors, mounted for example at the temples or at the sides of the head, either separate from the electrodes or built into the electrodes. The three impedance signals might be useful for independently obtaining estimates of global, left hemisphere and right hemisphere cerebral hemodynamic parameters, such as cerebral blood flow or cerebral blood volume.

FIG. 2C shows a system 230 for measuring an indication of a degree of cerebrovascular bilateral asymmetry in a subject 232, according to another embodiment of the invention. A PPG sensor 234 is placed on the subject's right temple, and a PPG sensor 236 is placed on the subject's neck, adjacent to a major artery on the right side of the neck, for example the right carotid artery 237. As seen reflected in mirror 220, a PPG sensor 238 is placed on the subject's left temple, and a PPG sensor 240 is placed on the neck of the subject adjacent to a major artery on the left side of the neck, for example the left carotid artery 241. Cables 242 connect the PPG sensors to a controller 244. System 230, unlike systems 100, 200, and 210, does not use impedance measurements at all, but only PPG sensors.

Controller 244 records PPG waveforms from all four sensors, as a function of time over one or more cardiac cycles. In general, it is expected that there is a phase delay between the rise in blood pressure in a carotid artery following the diastole, and the rise in the blood pressure in the smaller surface arteries of the head that the carotid artery feeds into. This phase delay can be measured, on each side of the head, by measuring the phase delay between the rise in the PPG signal from the PPG sensor adjacent to the carotid artery, and the rise in the PPG signal from the PPG sensor on the temple. Asymmetries in cerebral blood circulation in the two sides of the brain may affect the phase delays in the PPG signals from the two sides of the head. For example, a blocked artery inside the brain on one side of the head may result in greater surface blood flow on that side of the head, and a shorter phase delay on that side of the head, than on the other side. Other mechanisms may also affect the phase delay in the PPG signals.

Controller 244 analyzes the PPG waveforms from all four PPG sensors, to find the phase delay between the rise in signal at the carotid artery, and the rise in signal at the temple, on each side of the head. Controller 244 then uses the difference in phase delay on the two sides of the head, to estimate a degree of bilateral asymmetry in cerebral blood circulation.

As noted above, when using system 100, 200, 210, or 230 to detect and measure cerebrovascular asymmetries, it is primarily the impedance (or PPG) signal as a function of time, or as a function of phase of the cardiac cycle, during one or more cardiac cycles, that is of interest. Optionally, two waveforms are derived from two mirror image impedance signals, and/or two minor image PPG signals, over one or more cardiac cycles, using any of the methods described below, such as averaging the waveforms over a plurality of cardiac cycles, or using any method known in the art. Differences in characteristics of the two waveforms are used to determine the asymmetry. In some embodiments of the invention, waveforms generated from the two sides of the head are first analyzed separately, using any of the methods described below, or described in any of the related patent applications cited above, or using any method known in the art, thereby generating one or more measures reflecting characteristics of the two waveforms. The measures for the two waveforms are then compared, to provide an indication of a degree of cerebrovascular bilateral asymmetry. Alternatively or additionally, a third waveform is generated from a combination of the two waveforms, for example from a difference between them, or a ratio of them. The third waveform is then analyzed, to generate a measure of asymmetry, using any method described below or known in the art.

In some embodiments of the invention, not only are cerebrovascular asymmetries detected and measured, but, if an asymmetry is found, a determination is made on which side of the head a stroke or other pathology causing the asymmetry is likely to be located. This is done, for example, by comparing a waveform from impedance and/or PPG data, for each side of the head, to an expected or measured waveform obtained in a similar way from a healthy subject. The waveform that differs most from the waveform from a healthy subject is likely to be the one that is abnormal, and the pathology is likely to be found on the corresponding side of the head. Optionally, a degree of difference between the abnormal waveform and the waveform from a healthy subject is used to determine a degree of severity of the stroke or other pathology.

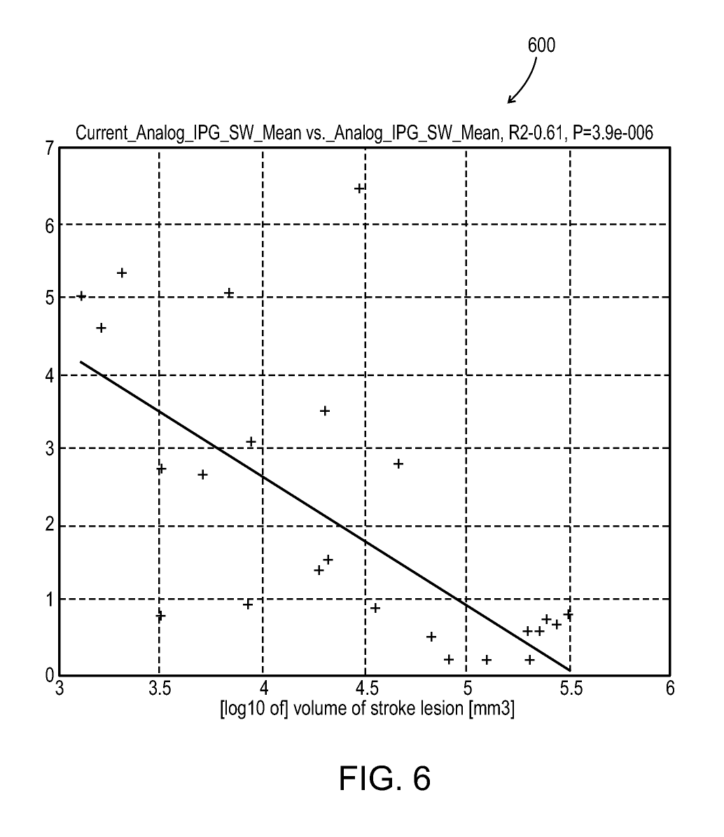

Optionally, the waveform is used to estimate in a quantitative way the severity of a stroke or other pathology. This is done, for example, by using the results of a study showing a correlation between a measure of the waveform, and a measure of severity of the pathology. An example is the correlation between normalized slow wave amplitude and volume of stroke lesion, shown below in FIG. 6.

In some embodiments of the invention, a degree of asymmetry, found by any of the methods described, is monitored in acute stroke patients, in a home or hospital setting, and medical personnel are alerted if the degree of asymmetry increases by 10%, or by 20%, or by 30% of the value it had when the patient was last examined, or if the degree of asymmetry crosses a pre-defined threshold value.

### Procedures for Analyzing the Signals

FIG. 3 shows a plot 300 of a cerebral impedance signal or a PPG signal, as a function of time, or a time parameter, for a cardiac cycle, and illustrates various ways of analyzing this signal, according to an exemplary embodiment of the invention. Optionally, the impedance signal is obtained using any of the electrode configurations described in FIG. 1, 2A or 2B, or in any of the related patent applications listed above, or with any electrode configuration known from the prior art for cerebral impedance measurements, and optionally the signal is pre-processed using any procedure described above or described in the related applications or in the prior art cited in the Background section or in other prior art, including combining signals from different pairs of electrodes, or combining a signal from a PPG sensor with an impedance signal, for example taking a difference between them, or a ratio of them. In some embodiments of the invention, PPG signals alone are used, without any impedance measurements, and it should be understood that, whenever impedance signals are mentioned here, PPG signals could be used instead.

It should be noted that the plot in FIG. 3 shows the systolic point, where the actual impedance of the head is lower because the blood volume in the head is higher, as higher on a y-axis 302, while the diastolic point is shown as lower on y-axis 302, as is conventional when plotting cerebral impedance. This is also true if the plot represents a PPG signal. As used herein, including in the claims, expressions such as “higher,” “lower,” “rising”, “falling”, positive or negative slope, positive or negative second or third derivative, and the like, will refer to the impedance signal (or PPG signal) as conventionally plotted, and not to the actual measured impedance.

Optionally, the signal plotted in FIG. 3, and analyzed, represents data from a single cardiac cycle, and the x-axis 304 is the actual time at which the data was taken. Alternatively, data from a plurality of cardiac cycles is combined, for example aligned and averaged together, and the signal in FIG. 3 represents such an average, and x-axis 304 represents a phase of the cardiac cycle with the dimensions of time, rather than actual time. The term “time parameter” as used herein covers both cases, and will sometimes loosely be referred to herein simply as “time,” while an interval of the time parameter may be referred to as an interval of time or a time interval, in cases where it will not cause any confusion to do so. A signal like that shown in plot 300, whether it represents a single cardiac cycle or an average over a plurality of cycles, is referred to herein as a “complex”.

Optionally, before the data from different cardiac cycles is aligned and averaged, it is detrended, so that the minimum value at the beginning and end of each cycle, corresponding to the diastolic phase, is always at a constant value, for example zero. Optionally, before detrending the data, the average value of the data from each cardiac cycle, or the minimum value from each cardiac cycle, is found and recorded, as one of the measures which is optionally used in the data analysis, including measurements of slow waves, as will be described below. Optionally, after the data is detrended, the diastolic points from the different cardiac cycles are aligned by aligning ECG signals, recorded from the subject when impedance measurements are made. Alternatively, the different cardiac cycles are aligned by aligning their minimum points, which are assumed to represent the diastolic point. Optionally, the length of each cardiac cycle is adjusted so the cycles are all the same length, before averaging, so that the final diastolic points are also all aligned.

Optionally, high frequency noise in the signal is filtered out, before and/or after aligning and averaging the different cycles. Optionally, individual cardiac cycles are checked for similarity to neighboring cycles, or previous cycles, or a running average of previous cycles, or a theoretically expected cycle, and cycles that are too different are removed, before averaging. Some ways of removing disparate cycles and averaging the data are described, for example, in the related PCT patent application PCT/IB2006/050174.

A complex like that shown in plot 300 can be analyzed in a number of ways, to generate various measures, some of which have been found to correlate substantially with various cerebral hemodynamic parameters of clinical interest. One of the measures, an average of the data or a minimum of the data over each cardiac cycle before detrending, has been mentioned above. The data before detrending can be used to determine the absolute dc impedance as well as to determine the amplitude and other characteristics of slow waves, typically at frequencies of 0.08 to 0.2 Hz. The amplitude of slow waves, normalized by the average impedance or by the peak-to-peak impedance over a cardiac cycle, has been found by the inventors to correlate well with the volume of ischemic stroke lesions, as determined by CT. An example of data showing such a correlation is given in FIG. 6, described below. In some embodiments of the invention, the amplitude of slow waves, optionally normalized, is monitored in acute stroke patients, with medical personnel alerted if the amplitude changes by 10%, or by 20%, or by 30%, particularly if the amplitude decreases, which indicates a worsening of the patient's condition, or if the amplitude falls below a pre-defined threshold value. The other measures, to be described below, are optionally found from the signal only after detrending, at which stage the signal generally resembles plot 300.

A minimum 306 in the signal of plot 300 marks the start diastole at the beginning of the cardiac cycle, and a minimum 308 marks the end diastole at the end of the cycle. The time interval 310 between time 312, when the start diastole occurs, and time 314, when the end diastole occurs, represents one cardiac cycle, and is sometimes used to normalize other time intervals in the signal.

### Measures of Rise Time

A maximum 316 in the signal, at time 318, marks the systole. A time interval 320 between time 318 of the systole, and time 312 of the start diastole, is called the total rise time, and may be divided by time interval 310 to obtain the normalized total rise time.

Noise in the signal may produce errors in the time of the minimum and the maximum, and hence in the total rise time 320. A more robust measure of the rise time may be obtained by instead taking a time interval beginning at a point 322 where the signal is a fixed percentage above the minimum, for example about 5%, about 10%, or about 20% of the way, or larger or smaller or intermediate percentages, from the minimum to the maximum, and ending at a point 324, where the signal is a fixed percentage below the maximum, for example about 10%, about 20%, or about 30%, or larger or smaller or intermediate percentages, below the maximum. These points occur at times 326 and 328 in FIG. 3, with the fixed percentage levels being 10% above the minimum and 20% below the maximum, and the difference between them represents a robust rise time 330. Because the beginning and end times of interval 330 usually do not occur near extrema of the signal, the length of interval 330 is less sensitive to noise than the length of interval 320.

It should be noted that, in the signal shown in FIG. 3, the data crosses a level 20% below the maximum first at point 324, then goes below this level after reaching a local maximum, and crosses the level again at point 332, at time 334. The robust rise time can be defined in different ways, with the end point of the interval either being the first time the signal crosses the fixed value (20% below the maximum in the case illustrated in FIG. 3), or the last time the signal crosses the fixed value before reaching maximum 316. The first definition yields robust rise time 330. The second definition would yield a longer robust rise time 336. If a first local maximum 338 is at a level below the fixed level, or if a local minimum 344 following local maximum 338 is above the fixed level, or if there is no local maximum 338 before maximum 316, then the signal will only cross the fixed value once, and these definitions will all yield the same robust rise time. The choice of fixed level, used in defining the end point of the robust rise time, may depend on what levels the first local maximum and the dip following the first local maximum typically occur at, for the signal being analyzed, and may also depend on whether the robust rise time is intended to be an approximation to the total rise time, or to the generally much shorter initial rise time to the first local maximum 338. For the signals analyzed by the inventors, and found to be correlated with clinical parameters as will be described below, the robust rise time has been defined so that the interval ends at the first point which is 20% below the maximum, such as point 324, and generally this point occurs before the first local maximum.

An interval 342 from time 312 of minimum 306, to a time 340 of first local maximum 338, may also be defined, and will be referred to herein as the rise time to the first local maximum. Optionally, the first local maximum is defined in such a way as to exclude a local maximum very close to the minimum, due to noise near the minimum. For example, a signal point is considered as a candidate to be the first local maximum only if it is sufficiently close to maximum 316, for example at least 50% of the way from minimum 306 to maximum 316, or at least 70% of the way. Optionally, it is also required that there be a long enough time interval surrounding the signal point, for example at least 10 msec or at least 20 msec, with all signal points in the time interval sufficiently close to maximum 316, in order to consider the point as a candidate to be the first local maximum.

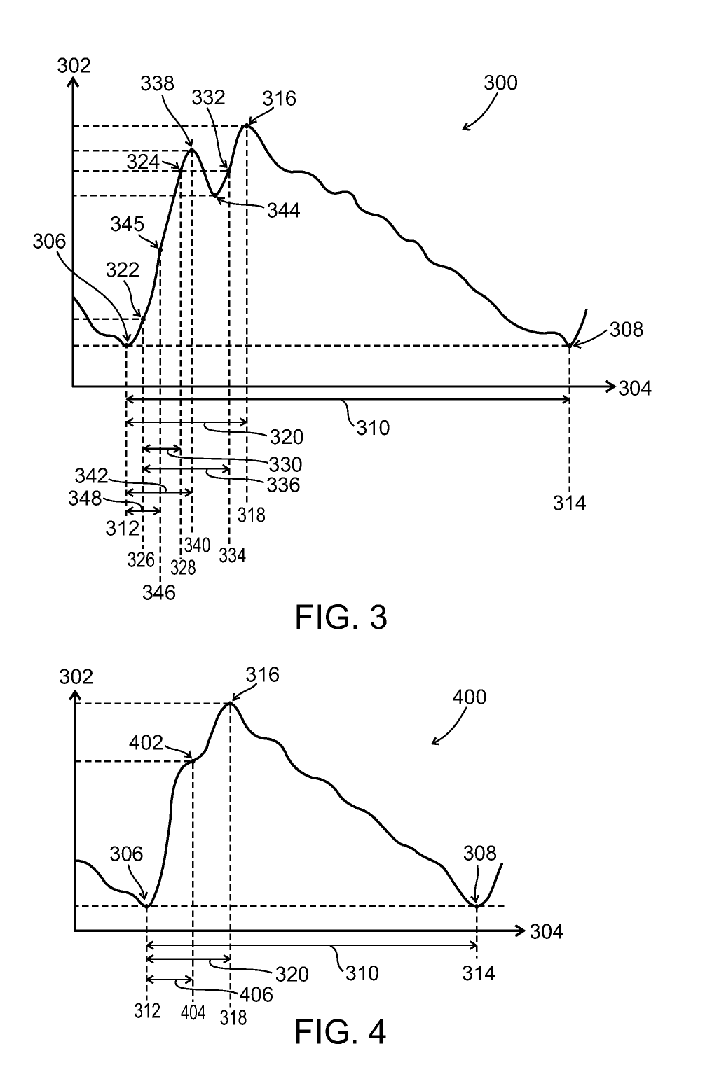

In some cases, the complex may not show a local maximum before maximum 316, but there may be an inflection point before maximum 316 which can play a role similar to a first local maximum, in analyzing the signal. FIG. 4, for example, shows a plot 400 of a complex, similar to plot 300 in FIG. 3, with axis 302 and axis 304 defined in the same way, and with a minimum 306 at time 312, a minimum 308 at time 314, and a maximum 316 at time 318. In FIG. 4, however, there is no first local maximum 338, but there is an inflection point 402, at time 404, with positive third derivative, at a level similar to first local maximum 338 in FIG. 3. Point 402 is referred to as a local effective maximum, a term which, as used herein, also includes an actual local maximum such as local maximum 338 in FIG. 3. Interval 406 between time 312 and time 404 is referred to herein as rise time to first local effective maximum. Optionally, the first local effective maximum, like the first local maximum, is defined in a way that excludes a point very close to the minimum, which is an inflection point with positive third derivative only due to noise. For example, a signal point is only a candidate if it is sufficiently close to maximum 316, for example at least 50% of the way or at least 70% of the way from minimum 306 to maximum 316, and optionally only if the point is surrounded by a long enough interval, for example at least 10 msec or at least 20 msec, which is also sufficiently close to maximum 316. Optionally, the point is only considered a candidate if it has a slope sufficiently small, for example, less than half of the slope at a point 344 where the slope is greatest, or less than half of the average slope between minimum 306 and that point.

As used herein, “first peak rise interval” is a generic term that includes both the rise time to first local maximum, and the rise time to first local effective maximum. As used herein, “rise interval” is a general term that includes first peak rise interval, total rise time, and robust rise time. Optionally, a rise interval, by any definition, is normalized by dividing it by cardiac cycle time interval 310.

The inventors have found relatively high linear correlations, for example R² from about 0.5 to 0.75, between the total rise time, or the robust rise time, normalized by the length of the cardiac cycle, and cerebral hemodynamic parameters, including regional cerebral blood volume, and regional cerebral blood flow, which can be independently measured by CT, or perfusion CT, and which provide a measure of the volume of an ischemic stroke lesion, with lower regional blood volume and blood flow indicating a more severe stroke. A shorter normalized total rise time, or robust rise time, is correlated to greater regional cerebral blood flow, and greater regional cerebral blood volume. Optionally, the normalized rise time for the impedance signal is divided by the normalized rise time for the PPG signal from one side of the head, which provides an even stronger correlation to regional cerebral blood flow and regional cerebral blood volume on that side of the head.

Furthermore, normalized robust rise time was found to have a high sensitivity (89% to 100%) and high specificity (60% to 75%) for predicting clinical symptoms of moderate to severe cortical stroke over the next 24 to 48 hours, in two small samples of patients. In some embodiments of the invention, the normalized total rise time or normalized robust rise time, is used to estimate regional cerebral blood flow and/or regional cerebral blood volume, or to diagnose the likely severity of strokes, in a clinical setting. In some embodiments of the invention, the normalized total rise time, or normalized robust rise time, is monitored in acute stroke patients, in a hospital or in a home setting, and medical personnel are alerted if it changes by more than 10%, or more than 20%, or more than 30%, particularly if it increases, indicating a worsening of the patient's condition. Optionally, medical personnel are alerted if the normalized total rise time, or normalized robust rise time, goes above a pre-defined threshold value. Optionally, the normalized total rise time, or normalized robust rise time, that is being monitored, is adjusted using the PPG signal, as described above.

### Other Measures of Signal as Function of Cardiac Phase

Some other measures which are optionally used to analyze a signal as a function of cardiac phase include the maximum slope, at point 345 in FIG. 3 at time 346; the height of maximum 316, measured relative to minimum 306; the maximum slope normalized to the height of maximum 316; the length of an interval 348 from time 312 of the minimum to time 346 of the maximum slope; the ratio of first peak rise interval to total rise time; the ratio of robust rise time to total rise time, particularly if the robust rise time is defined so that it is similar to the first peak rise interval; and the ratio of the height of the first local effective maximum (point 338 in FIG. 3, or point 402 in FIG. 4), to the height of maximum 316, both heights measured relative to minimum 306.

Sometimes there is a second local maximum, before absolute maximum 316. In some embodiments of the invention, the height of the second maximum is used in the measures described above, whether or not it is also the absolute maximum of the complex. For example, the height of first local effective maximum is optionally normalized to the height of the second maximum, whether or not that is also the absolute maximum 316.

It should be noted that measures that depend on an absolute height of the signal, for example the height of maximum 316, or the un-normalized maximum slope, rather than on dimensionless ratios of two heights, may be sensitive to the exact location of the electrodes and other variables that are difficult to keep constant from one set of measurements to another, and may be most meaningful when used to track changes over time when continuously monitoring a given subject.

### Measures Involving Integrals of Signal

Other measures used for analyzing a complex involve integrating the signal over the cardiac cycle interval 310, or over a portion of it, for example over total rise time 320. The signal as a function of the time parameter in a complex, such as the signals shown in FIGS. 3 and 4, is typically approximately triangular, rising roughly linearly from the start diastole to the systole, and falling roughly linearly from the systole to the end diastole. The integral of this signal over a cardiac cycle, with the mimimum set at zero, will be roughly half of the height of the maximum times the length of the cardiac cycle. Similarly the integral over the total rise interval will be roughly half the height of the maximum times the length of the total rise interval. Dividing the integral by the length of the cardiac cycle, in the first case, or by the length of the total rise time in the second case, gives an average value for the signal during the cardiac cycle, or during the total rise interval, which in both cases will be roughly half of the maximum. So either of these measures, multiplied by two, provides an approximate substitute for the height of the maximum, which may be more robust, being less affected by noise, than the actual height of the maximum. This average value of the signal may be used as a more robust substitute for the height of the maximum, for estimating cerebral hemodynamic parameters that have been found to be correlated with the height of the maximum. Examples of such parameters may include intracranial pressure and cerebral blood volume.

Similarly, if the integral is taken over the rise time 342 to the first local maximum (or the first local effective maximum) and it is divided by the rise time 342 to the first local maximum, then the result, the average value of the signal during the rise to the first local maximum, will be approximately half the height of the first local maximum, if the rise is to the first local maximum is nearly linear. This measure can be used as a more robust substitute for the height of the first local maximum.

In some embodiments of the invention, a complex is analyzed by taking a ratio of the integral over the total rise time, or the integral over the robust rise time, or the integral over the rise time to the first local maximum, to the integral over the length of the cardiac cycle. These ratios may be similar to the ratio of total rise time, or robust rise time, or rise time to first local maximum, to the length of the cardiac cycle, discussed above, and have also been found to be correlated with regional cerebral blood flow and regional cerebral blood volume. A particularly strong correlation has been found to regional cerebral blood volume when the ratio of integrals for the impedance signal is divided by the ratio of integrals for the PPG signal. Any of these quantities may be monitored in acute stroke patients, alerting medical personnel if they change, particularly if they increase, by 10%, or by 20%, or by 30%, or if they rise above a pre-defined threshold value.

The ratio of the integral of the signal over the robust rise time, to the integral over the length of the cardiac cycle, has been found by the inventors to be an especially useful quantity for analyzing some impedance and PPG signals, particularly with the robust rise time defined as starting when the signal is 10% above the minimum, and ending when it is 20% below the maximum. Correlations between this quantity, and clinically useful parameters in ischemic stroke patients, have been found to be between 0.5 and 0.75, for some signals, in a clinical study, as will be described below in the section “Observed Correlations with Clinical Parameters.”

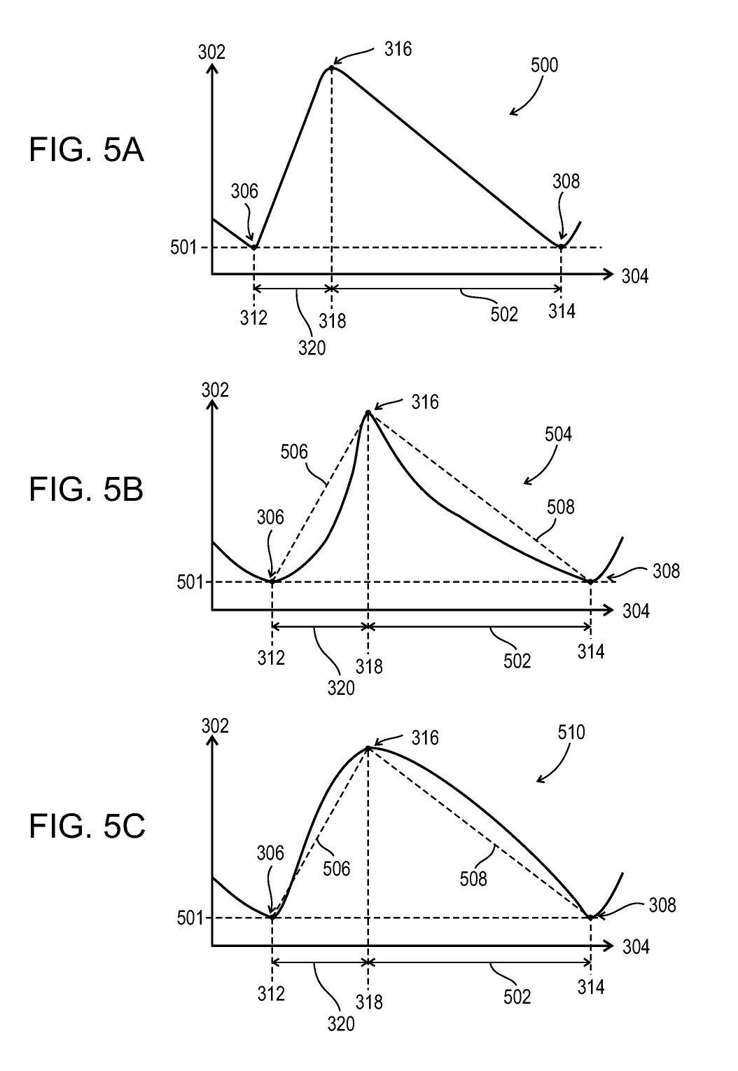

In some embodiments of the invention, a complex is analyzed by taking a ratio between the height of the maximum as estimated from the integral divided by the time interval, and the actual height of the maximum. That is to say, an integral of the signal is taken, either over the total cardiac cycle, or just over the total rise interval, and is divided by the integration time and by the height of the maximum. This ratio is a measure of the concavity or convexity of the signal as it rises and falls. When the integral is taken over the whole cardiac cycle, or over the total rise interval, this ratio is similar to the reciprocal of the Pulsatility Index measured in Ultrasound TCD waveforms. FIGS. 5A-5C schematically illustrate complexes for which this ratio has different values. In FIG. 5A, the complex is very nearly triangular, with the signal rising linearly to its maximum, and falling linearly to the minimum. For FIG. 5A, the ratio will be close to 0.5. In FIG. 5B, the signal is concave, rising slowly at first and then more rapidly to its maximum, and falling rapidly at first and then more slowly to the minimum. For FIG. 5B, the ratio will be less than 0.5. In FIG. 5C, the signal is convex, rising rapidly at first and then more slowly to its maximum, and falling slowly at first and then more rapidly to the minimum. For FIG. 5C, the ratio will be more than 0.5, though less than 1. The ratio will characterize an average concavity or convexity over the whole cardiac cycle, or just over the rising or falling portion, depending on whether the integral is taken over the whole cardiac cycle, or just over the rising or falling portion. The integral can also be taken from the minimum to the first local maximum, with the ratio characterizing an average concavity or convexity over the rise time to the first local maximum.

Optionally, any of the measures, if found for single cardiac cycles, are smoothed over time, to eliminate artifacts that may produce noisy results for some cardiac cycles. Even a measure that is found from an average of aligned data from a plurality of cardiac cycles, as described above, may be smoothed over time, with each calculated value of the measure representing a different set of cardiac cycles, for example.

Optionally, any of the methods of analyzing an impedance signal described above are used for analyzing two mirror image asymmetric impedance signals, obtained using three or more electrodes placed symmetrically on the head, as described above. For example, the values of one or more cerebral hemodynamic parameters are estimated, using any of the methods described above, for each of the asymmetric impedance signals. Comparing these values, and/or comparing changes in the values over time, may provide information on asymmetries in the cerebral hemodynamic parameters, which may be useful for diagnosing and/or monitoring strokes and other cerebrovascular conditions. Alternatively, the two impedance signals are combined in some way, for example taking a difference between them, or a ratio. The combined signal is then analyzed according to any of the methods described above, to find measures that are characteristic of differences between the left and right sides of the head. These measures may be useful for diagnosing and/or monitoring strokes and other cerebrovascular conditions.

Observed Correlations with Clinical Parameters

FIG. 6 shows a plot 600 of the volume of the stroke lesion, measured by CT, in a group of 25 ischemic stroke patients, on the horizontal axis, and a normalized slow wave amplitude on the vertical axis. Both the horizontal and vertical scales are logarithmic. The slow wave amplitude is from an impedance signal taken across the head, with electrodes in positions similar to electrodes 204 and 206 in FIG. 2, and is normalized to an average of the impedance signal. The square of the correlation is R²=0.61. The correlation is negative, with lower slow wave amplitude corresponding to large stroke lesion volume. Almost as high a correlation was found when the slow wave amplitude was normalized to the average peak-to-peak impedance over a cardiac cycle. The data from five patients, out of an original group of 30 patients, was eliminated from the analysis, because two of the patients did not fit the study clinically, two of the patients were outliers due to poor signal quality, and one patient had zero stroke volume which could not be fitted on the logarithmic scale.

A clinical study was performed, using stroke patients, in which certain standard cerebral hemodynamic parameters were measured by perfusion CT, and were also estimated using various dimensionless measures based on IPG and PPG signals as functions of cardiac cycle phase. The IPG electrodes placed at the left and right corners of the forehead, and a PPG sensor was placed on each temple. The IPG signals were found using up to 1 mA of current, at about 25 kHz. The signals were detrended, setting their minimum for each cardiac cycle to the same level, and in some but not all cases several consecutive cardiac cycles were averaged together, in phase, to reduce noise while retaining the shape of the signal as a function of cardiac cycle phase. A best linear fit and correlation were calculated for the dimensionless measures based on the IPG and PPG signals, and the values of the parameters measured by perfusion CT. Correlations found ranged from approximately 0.5 to 0.7, with values of the parameters generally ranging over a factor of about 2 or 3, or occasionally more, for the different patients in the sample. The best linear fits listed here could be used as a starting point for providing estimates of these cerebral hemodynamic parameters from IPG and PPG data. Standard units were used for the parameters: milliliters per 100 grams of tissue for CBV, milliliters per 100 grams of tissue per minute for CBF, and seconds for TTP.

1) This measure was the ratio of a measure based on the IPG signal across the head, to a measure based on the PPG signal on the side of the head opposite to the stroke. For each of these signals, the measure was a rise time interval starting at the diastolic point, and ending at the point of maximum slope. This measure was used to estimate the parameter hemispheric CBV on the stroke side. The correlation was R²=0.54, and the best linear fit was:

Measure=Parameter/4.8+0.06

2) This measure was the ratio of a measure based on the IPG signal across the head, to a measure based on the PPG signal on the same side of the head as the stroke. For each of these signals, the measure was the integral of the signal over the robust rise time, normalized to the integral of the signal over the whole cardiac cycle. This measure was used to estimate the parameter global CBV. The correlation was R²=0.72, and the best linear fit was:

Measure=−Parameter/6.9+1.49

3) This measure was the ratio of a measure based on the IPG signal across the head, to a measure based on the PPG signal on the opposite side of the head from the stroke. For each of these signals, the measure was the normalized integral of the signal over the robust rise time, defined above. This measure was used to estimate the parameter global CBV. The correlation was R²=0.59, and the best linear fit was:

Measure=−Parameter/8.3+1.4

4) This measure was the normalized integral of the signal over the robust rise time, defined above, for the PPG signal on the same side of the head as the stroke. This measure was used to estimate hemispheric CBF on the same side of the head as the stroke. The correlation was R²=0.56, and the best linear fit was:

Measure=Parameter/650+0.12

5) This measure was the normalized integral of the signal over the robust rise time, defined above, for the PPG signal on the same side of the head as the stroke. This measure was used to estimate hemispheric TTP on the same side of the head as the stroke. The correlation was R²=0.56, and the best linear fit was:

Measure=Parameter/420+0.08

6) This measure was the normalized integral of the signal over the robust rise time, defined above, for the IPG signal across the head. This measure was used to estimate global TTP. The correlation was R²=0.46, and the best linear fit was:

Measure=Parameter/280+0.04

7) This measure was a normalized rise time curvature of the signal, for the PPG signal on the same side of the head as the stroke. This normalized rise time curvature is defined by first fitting the signal during the robust rise time to a straight line, then fitting the signal during the robust rise time to a parabola, and taking the difference in the cardiac phase, or time, where the two fits cross a level halfway between the minimum and maximum of the signal. This difference is then normalized to the robust rise time. This measure was used to estimate the ratio of regional CBF on the same side of the head as the stroke, to global CBF, a quantity with a range of about a factor of 8 over the patients in the sample. The correlation was R²=0.53, and the best linear fit was:

Measure=Parameter/21.6+0.017

### Clinical Uses of Impedance Measurements

Any of the measures that show a strong correlation with regional cerebral blood volume and regional cerebral blood flow, for example normalized robust rise time and related measures, may be used in a number of ways in clinical settings. For example, impedance measurements may be made on acute stroke victims by emergency medical personnel, to distinguish ischemic strokes from hemorrhagic strokes, as well as from other causes of neurological symptoms such as tumors, and to evaluate the extent of ischemic strokes. This information may be used to help decide which patients are likely to benefit from thrombolytic therapy, which generally has a narrow window of opportunity for administering it. Patients are generally considered likely to benefit from thrombolytic therapy only if they have ischemic strokes falling under a certain level of severity. For patients with small clinical manifestation or larger stroke lesions, the likely benefits of thrombolytic therapy may be outweighed by an increased risk of cerebral hemorrhage.

Measures correlated with regional cerebral blood volume and blood flow may also be used to monitor patients during and following therapy that affects cerebral circulation, such as ventilation, thrombolytic therapy, and therapy for reducing blood pressure. By monitoring a patient in real time during the administration of such therapy, the total dose of a therapeutic agent or the rate at which the therapy is administered could be adjusted, depending on the response of the patient. After the therapy is administered, the patient can be monitored for conditions that require immediate intervention. For example, if cerebral blood flow suddenly decreases, that could indicate a new blood clot, which might be located with a cerebral angiograph and removed mechanically. Too great a rise in cerebral blood flow, in any stroke patient, might indicate a dangerous rise in blood pressure, which could be countered with appropriate medication. Finally, impedance measurements could indicate when regional cerebral blood flow has returned to normal and/or stabilized, and the patient can be sent home. Because impedance monitoring can be done continuously, it may provide a better indication of the stability of regional cerebral blood flow over time, than methods such as CT or MRI, which may be more accurate, but cannot be done continuously.

Measures of slow wave amplitude, because they are correlated with stroke volume in ischemic stroke patients, may also be used by emergency medical personnel to evaluate the severity of the strokes of such patients, to determine which patients are likely to benefit from thrombolytic therapy.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of finding an indication of a degree of cerebro-vascular bilateral asymmetry in a subject, comprising:
a) measuring a first impedance waveform and a second impedance waveform of the subject's head as functions of time, in each case by finding a potential difference between two voltage electrodes associated with passing a given injected current through the head between at least two current electrodes, wherein in each case the voltage electrodes are located asymmetrically on the head with respect to the bilateral symmetry plane of the head, or the current is injected asymmetrically into the head, or both, and wherein the locations of the voltage electrodes and the distribution of current injection in measuring the second impedance waveform are mirror images, with respect to the bilateral symmetry plane of the head, of what they are in measuring the first impedance waveform; and
b) finding the indication of the degree of bilateral asymmetry from a difference between characteristics of the first and second impedance waveforms;
wherein the characteristic of the first impedance waveform comprises a ratio of a height of a first peak to a height of a second peak, relative to a minimum, of the first impedance waveform or a waveform derived from it, and the characteristic of the second impedance waveform comprises a ratio of a height of a first peak to a height of a second peak, relative to a minimum, of the second impedance waveform or a waveform derived from it.

2. A method according to claim 1, wherein the electrodes used for measuring the first and second impedance waveforms comprise at least three electrodes, and the method also includes placing the at least three electrodes on the head in a bilaterally symmetric configuration before measuring the first and second impedance waveforms.

3. A method according to claim 2, wherein placing the at least three electrodes on the head comprises placing a first electrode and a second electrode symmetrically on the left and right sides of the head, and placing a third electrode substantially at the bilateral symmetry plane of the head.

4. A method according to claim 3, wherein measuring the first impedance waveform comprises measuring a potential difference between the first and third electrodes when current is passed between the first and third electrodes, and measuring the second impedance waveform comprises measuring a potential difference between the second and third electrodes when current is passed between the second and third electrodes.

5. A method according to claim 3, wherein measuring the first impedance waveform comprises measuring a potential difference between the first and third electrodes when current is passed between the first and second electrodes, and measuring the second impedance comprises measuring a potential difference between the second and third electrodes when current is passed between the first and second electrodes.

6. A method according to claim 2, wherein the at least three electrodes comprise at least first, second, third and fourth electrodes, and placing the electrodes on the head comprises placing the first and second electrodes symmetrically on the left and right sides of the head, respectively, and placing the third and fourth electrodes symmetrically on the left and right sides of the head, respectively, closer together than the first and second electrodes.

7. A method according to claim 6, wherein the first impedance waveform is measured using the first and fourth electrodes, and the second impedance waveform is measured using the second and third electrodes, also including:
a) measuring a first surface impedance waveform using the first and third electrodes;
b) measuring a second surface impedance waveform using the second and fourth electrodes; and
c) correcting the first and second impedance measurements, to reduce a contribution of surface impedance, using the results of the first and second surface impedance measurements.

8. A method according to claim 1, wherein measuring the first impedance waveform comprises finding the potential difference between a first voltage electrode placed on a temple of the subject, and a second voltage electrode placed on the head behind the ear, on a same side of the head as the first voltage electrode.

9. A method according to claim 8, wherein measuring the first impedance waveform comprises finding the potential difference between the first and second voltage electrodes while passing the current through the head between a first and a second current electrode, the first current electrode being comprised in a same structure as, or placed adjacent to, the first voltage electrode, and the second current electrode being comprised in a same structure as, or placed adjacent to, the second voltage electrode.

10. A system for finding an indication of a degree of cerebrovascular bilateral asymmetry, comprising:
a) an electric current source;
b) a voltmeter adapted to measure potential differences between two electrodes;
c) a set of at least three electrodes, at least three of them adapted to pass current from the current source through the head, and at least three of them, the same as or partly or completely different than the electrodes adapted to pass current, adapted to be used by the voltmeter for measuring a potential difference between different locations on the head; and
d) a controller which, when the electrodes are placed appropriately on the head, is adapted to make a first impedance measurement by using a first subset of the electrodes, placed asymmetrically with respect to the bilateral symmetry plane of the head, to measure the voltage associated with a given current passed through the head, and to use a second subset of the electrodes to make a second impedance measurement that is a mirror image of the first impedance measurement with respect to the bilateral symmetry plane of the head, and to use a difference between characteristics of waveforms of the first and second impedance measurements to find the indication of the degree of bilateral symmetry, by finding a ratio of a height of a first peak to a height of a second peak, relative to a minimum, for the first impedance waveform, or a waveform derived from the first impedance waveform, or both, and by finding a ratio of a height of a first peak to a height of a second peak, relative to a minimum, for the second impedance waveform, or a waveform derived from the second impedance waveform, or both.

11-38. (canceled)

39. A method of finding an indication of a degree of cerebrovascular bilateral asymmetry in a subject, comprising:
a) measuring a characteristic of surface blood flow on the left side of the subject's head, using at least a first sensor in a region on the left side of the head, the characteristic comprising a ratio of a height of a first peak to a height of a second peak, relative to a minimum, for a waveform of a signal of the first sensor as a function of time;
b) measuring a characteristic of surface blood flow on the right side of the subject's head, using at least a second sensor in a region on the right side of the head, the characteristic comprising a ratio of a height of a first peak to a height of a second peak, relative to a minimum, for a waveform of a signal of the second sensor as a function of time; and
c) using a difference between the characteristics of the surface blood flows on the left and right sides of the head to find the indication of the degree of cerebrovascular bilateral asymmetry.

40. A method according to claim 39, wherein the first and second sensors are PPG sensors.

41. A method according to claim 39, wherein the first and second sensors are surface impedance sensors.

42-43. (canceled)

44. A method according to claim 39, also including measuring a value of a cerebral hemodynamic parameter symmetrically across the head, wherein using the difference between characteristics of the surface blood flows on the left and right sides of the head comprises correcting the value of the cerebral hemodynamic parameter using the surface blood flow on the left side, correcting the value of the cerebral hemodynamic parameter using the surface blood flow on the right side, and using a difference between the two corrected values of the cerebral hemodynamic parameter.

45. A method according to claim 39, wherein the first and second sensors are substantially identical, and the regions on the left and right sides of the head are substantially mirror images of each other around the bilateral symmetry plane of the head.

46. A system for finding an indication of a degree of cerebrovascular bilateral asymmetry, comprising:
a) a first and a second sensor adapted for measuring surface blood flow on the head; and
b) a controller adapted to use the first and second sensors to measure characteristics of surface blood flow in regions respectively on the left and right sides of the head, and to use a difference between the characteristics of the measured blood flows on the left and right sides of the head to find the indication of the degree of cerebrovascular bilateral asymmetry, the characteristic of the blood flow on the left side of the head comprising a ratio of a height of a first peak to a height of a second peak, relative to a minimum, for a waveform of a signal of the first sensor as a function of time, and the characteristic of the blood flow on the right side of the head comprising a ratio of a height of a first peak to a height of a second peak, relative to a minimum, for a waveform of a signal of the second sensor as a function of time.

47. A method according to claim 1, wherein finding the indication of the degree of asymmetry comprises analyzing the first and second impedance waveforms, or a waveform derived from the first and second impedance waveforms, or both, by:
a) determining a minimum of the signal over the cardiac cycle time;
b) determining an effective maximum of the signal over the cardiac cycle time;
c) determining a rise interval of the cardiac cycle time, between the minimum and the effective maximum, over which the signal is rising according to a rise time criterion; and
cerebral blood volume, time to peak, and mean transit time, in a patient.

48. (canceled)

49. A method according to claim 1, wherein finding the indication of the degree of asymmetry comprises finding a peak-to-peak height of the first and second impedance waveforms, or a waveform derived from the first and second impedance waveforms, or both.

50. A method according to claim 1, wherein finding the indication of the degree of asymmetry comprises finding a maximum slope of the first and second impedance waveforms, or a waveform derived from the first and second impedance waveform, or both.

51. A method according to claim 1, wherein finding the indication of the degree of asymmetry comprises finding an interval from a time of minimum value, to a time of maximum slope, for the first and second impedance waveforms, or a waveform derived from the first and second impedance waveforms, or both.

52. (canceled)

53. A method according to claim 1, also comprising comparing the first and second impedance waveforms to an impedance waveform of a healthy subject, and determining which side of the head an abnormality causing the asymmetry is located on, using differences between the first and second waveforms, and the waveform of the healthy subject.

54. A method according to claim 1 or claim 39, wherein the indication of the degree of cerebrovascular bilateral asymmetry comprises a measure of severity of a pathological cerebrovascular condition.

55-59. (canceled)

60. A method according to claim 39, wherein the indication of the degree of cerebrovascular bilateral asymmetry comprises a measure of severity of a pathological cerebrovascular condition.
